(19) United States
(12) Patent Application Publication
(10) Pub. No.: US 2011/0004414 A1
(43) Pub. Date: Jan. 6, 2011

(54) Method for Predicting Respiratory Toxicity of Compounds

(75) Inventor: James M. McKim, Kalamazoo, MI (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: CEETOX, INC., Kalamazoo, MI (US)

(21) Appl. No.: 12/795,525

(22) Filed: Jun. 7, 2010

Related U.S. Application Data
(60) Provisional application No. 61/184,794, filed on Jun. 6, 2009.

(51) Int. Cl. G01N 33/48; G06F 19/00

(52) U.S. Cl. 702/19

(57) ABSTRACT

The invention provides methods for analyzing and predicting the in vivo respiratory toxicity of a compound (e.g., pharmaceutical, biological, cosmetic, or chemical compounds) or composition comprising a combination of an in vitro mammalian cell model with multiple endpoint analysis, and time and concentration response curves. The methods allow the determination of a predicted in vivo respiratory toxicity value of a compound without the use of animals, with a high degree of accuracy. The methods comprise detecting any combination of cell viability markers and expression levels of genes implicated in respiratory toxicity and/or sensitization, such as pro-inflammatory response genes, combining the viability and gene expression level data with concentration response and time response data, conducting a computational analysis, and comparing test compound data to a database of known respiratory toxicants/sensitizers to predict and/or analyze the respiratory toxicity. An indication of organ specificity is provided by a toxicity index, which is determined by comparing mean IC₅₀ values in lung cells to mean IC₅₀ values in liver cells.

### CROSS-REFERENCE TO RELATED APPLICATIONS

This application claims the benefit of U.S. Provisional Application No. 61/184,794, filed on Jun. 6, 2009, which is hereby incorporated by reference in its entirety.

### FIELD OF THE INVENTION

The invention relates to in vitro methods for detecting and/or predicting in vivo respiratory toxicity of a compound. The invention also relates to methods of screening chemicals, pharmaceutical drugs, cosmetics and/or candidate therapeutic treatments (e.g., small molecule and/or biological drugs) for respiratory toxicity. Further the invention provides methods for categorizing compounds into various classes of respiratory toxicity. The invention further relates to kits comprising reagents and directions for performing the various methods of the invention.

### BACKGROUND

There is a growing need for new in vitro alternatives to animal toxicity testing in both the chemical and personal care industries. The registration of new chemicals under REACH as well as Amendment VII to the Cosmetics Directive in Europe requires the development, validation, and utilization of new in vitro methodologies. Further, the use of animal models to assess the toxicity of a substance is costly and time consuming.

A common approach to solving the toxicology data deficit has been to incorporate in vitro toxicity testing of drugs candidate compounds into the drug discovery process at a time when candidate compounds are being identified for potency and efficacy against therapeutic targets. Quality toxicity data at this early stage permits pharmaceutical chemists to attempt to “design out” toxicity while maintaining efficacy/potency. Although this is a good idea in principle, in practice it has been extremely difficult to develop robust in vitro toxicity data and to match in vitro data with in vivo toxicity. (See, e.g., U.S. Pat. No. 6,998,249; U.S. patent application Ser. No. 12/339,826, filed Mar. 6, 2009, both incorporated by reference).

Key issues in developing these in vitro systems include determining the type and nature of assays to be utilized and the test system (cell types) to be employed. There are many biochemical and molecular assays that claim to assess toxicity in cells grown in culture. However, when a limited number of assays (e.g., one or two) are used over a limited range of exposure concentrations, the probability of false negative and false positive data is high. Some of the most commonly used assays include, but are not limited to, leakage of intracellular markers as determined by lactate dehydrogenase (LDH), glutathione S-transferase (GST), and potassium, and the reduction of tetrazolium dyes such as MTT, XTT, Alamar Blue, and INT. All have been used as indicators of cell injury. In all cases, the assays typically involve the use of one or two concentrations. These tests have been performed using lung epithelial cells obtained by lavage, or cell lines of lung origin. The resulting data provides a yes/no or live/dead answer. This minimalist approach to the toxicity-screening problem has resulted in little progress towards developing a robust screening system capable of providing a useful toxicity profile that has meaning for predicting similar toxicity in animals. Therefore, there remains a need in the art for the development of new screening systems that provide more useful toxicity information, especially toxicity information that can be obtained rapidly and cost-effectively at early stages of the drug discovery process, or to assess the risk of respiratory damage due to chemical exposure in the work place. As such, a need exists for toxicity screening systems that do not require the use of animals, but that provide reliable information on relative toxicity, mechanism of toxicity, and that effectively predict in vivo toxicity.

The ability to accurately and precisely identify respiratory toxins is of great importance to many industries, such as the pharmaceutical, personal care (e.g., cosmetics), and chemical industries. Current methods that are commonly used to analyze the respiratory toxicity of a substance typically include the use of an animal model system. The proposed ban on costly and time consuming animal studies for the personal care industry in Europe requires that new in vitro methodologies be developed.

Such in vitro models should be accurate and allow for the testing of multiple compounds, compositions, and mixtures, regardless of a compound's solubility. Also, one needs to determine not only whether a compound is toxic, but to understand the level of toxicity and how the test compound is inducing the toxic response. A robust multiple-endpoint analysis combined with a broad exposure concentration range, and a test system that accurately mimics the respiratory environment is crucial to increase accuracy of the model and to reduce both false positives and false negatives. Recent progress in the area of human three-dimensional cell models of respiratory origin provide has resulted in a physiologically relevant cell model.

Accordingly, there is a need in the art for improved methods that reliably assess the respiratory toxicity of a test substance (e.g., pharmaceutical, biological, cosmetic, and chemical compounds), without the use of animal models. Moreover, there is a need for methods that provide an extrapolation of in vitro data to a predicted in vivo minimum exposure level that would produce toxicity in the respiratory system. Thus, in vitro cell-based models able to predict toxicity specific to the respiratory system would be of considerable value in early drug, cosmetic and other product development.

While the cells, marker genes and individual endpoints in the present invention have been described previously, the combination of the cell model, the measurement of the expression of one or more marker genes, the monitoring of multiple endpoints and the computational analysis is a novel aspect of the invention.

### SUMMARY OF THE INVENTION

In a first aspect, the invention provides a method for predicting the in vivo respiratory toxicity of a compound, comprising: (a) culturing mammalian cells; (b) contacting the mammalian cells with a concentration of the compound; (c) measuring the expression level of one or more marker genes in the mammalian cells after contacting the cells with the compound; (d) monitoring multiple endpoints of cell viability and general cell health; (e) conducting a computational analysis of the concentration of the compound used to contact the cells, and the measured expression level(s) of the one or more marker genes; and (f) determining a predicted in vivo respiratory toxicity value based on the computational analysis.

In a second aspect, the invention provides a method for screening a compound for in vivo respiratory toxicity comprising: (a) culturing mammalian cells; (b) contacting the mammalian cells with a concentration of the compound; (c) measuring the expression level of one or more marker genes in the mammalian cells after contacting the cells with the compound; (d) monitoring multiple endpoints of cell viability and general cell health after contacting the cells with the compound; (e) conducting a computational analysis of the concentration of the compound used to contact the cells, and the measured expression level(s) of the one or more marker genes; (f) determining a predicted in vivo toxicity value based on the computational analysis; and (g) determining whether the toxicity value of the compound falls within acceptable limits for the particular in vivo use.

In a third aspect, the invention provides a method for categorizing the in vivo respiratory toxicity of a compound, comprising: (a) culturing mammalian cells; (b) contacting the mammalian cells with a concentration of the compound; (c) measuring the expression level of one or more marker genes in the mammalian cells after contacting the cells with the compound; (d) monitoring multiple endpoints of cell viability and general cell health after contacting the cells with the compound; (e) conducting a computational analysis of the concentration of the compound used to contact the cells and the measured expression level(s) of the one or more marker genes; and (f) determining a predicted in vivo toxicity value based on the computational analysis; wherein the computational analysis comprises a comparison of the data from the compound with data gathered from at least two compounds with known respiratory toxicity profiles, wherein the each of the two compounds with known respiratory toxicity profiles are classified independently as a respiratory sensitizer, a respiratory irritant, or a respiratory corrosive, wherein the at least two compounds are not members of the same toxicity profile class.

In a fourth aspect, the invention provides kits comprising at least a portion of the necessary reagents for conducting the methods described herein, and instructions for proper use of the kit. In certain embodiments, the kit of the invention comprises all the necessary reagents for conducting the methods described herein.

These aspects of the invention, as well as other aspects and embodiments will become apparent to those of skill in the art from the following detailed description of the invention.

### BRIEF DESCRIPTION OF THE DRAWINGS

FIG. 1-1A depicts a general schematic of the three dimensional EPIAIRWAY cell culture system (MatTek, Ashland, Mass.). 1B depicts the structure of human bronchiole tissue (10× resolution). 1C depicts the structure of EPIAIRWAY three dimensional airway model (10× resolution).

FIG. 2 shows the effect of bleomycin on cell structure (FIG. 2A); cell viability (FIG. 2B); cellular glutathione (GSH) (FIG. 2C); and expression of pro-inflammatory response genes CYP1A1, Bax, Bcl2, TNFα, TGFβ, IL-1a, IL-6 and IL-8 (FIG. 2D). For cell viability and cellular GSH, the results are expressed as mean % of control (vehicle) of three replicates and the error bars represent ±S.E.M. For RT-PCR analysis, data are expressed as mean fold induction over control (vehicle); housekeeping genes were analyzed and the most stable genes across all doses were used to generate a normalization factor that all samples were normalized to.

FIG. 3 shows the effect of bleomycin on lactate dehydrogenase (LDH) release by EPIAIRWAY cells. Data are expressed as mean % of control (vehicle) of three replicates. The error bars represent ±S.E.M.

FIG. 4 shows the effect of cadmium chloride on cell structure (FIG. 4A); cell viability (FIG. 4B); cellular glutathione (GSH) (FIG. 4C); and expression of pro-inflammatory response genes CYP1A1, CYP1A2, iNOS, Bax, Bcl2, TNFα, TGFβ, IL-1a, IL-6 and IL-8 (FIG. 4D). For cell viability and cellular GSH, the results are expressed as mean % of control (vehicle) of three replicates and the error bars represent ±S.E.M. For RT-PCR analysis, data are expressed as mean fold induction over control (vehicle); housekeeping genes were analyzed and the most stable genes across all doses were used to generate a normalization factor that all samples were normalized to.

FIG. 5 shows the effect of beryllium on cell viability (FIG. 5A); cellular glutathione (GSH) (FIG. 5B); and expression of pro-inflammatory response genes CYP1A1, Bax, Bcl2, TNFα, TGFβ, IL-1a, IL-6 and IL-8 (FIG. 5C). For cell viability and cellular GSH, the results are expressed as mean % of control (vehicle) of three replicates and the error bars represent ±S.E.M. For RT-PCR analysis, data are expressed as mean fold induction over control (vehicle); housekeeping genes were analyzed and the most stable genes across all doses were used to generate a normalization factor that all samples were normalized to.

FIG. 6 shows the effect of silica on cell viability (FIG. 6A); cellular glutathione (GSH) (FIG. 6B); and expression of pro-inflammatory response genes CYP1A1, Bax, Bcl2, TNFα, TGFβ, IL-1a, IL-6 and IL-8 (FIG. 6C). For cell viability and cellular GSH, the results are expressed as mean % of control (vehicle) of three replicates and the error bars represent ±S.E.M. For RT-PCR analysis, data are expressed as mean fold induction over control (vehicle); housekeeping genes were analyzed and the most stable genes across all doses were used to generate a normalization factor that all samples were normalized to.

FIG. 7 shows the effect of lipopolysaccharide (LPS) on cell viability (FIG. 7A); cellular glutathione (GSH) (FIG. 7B); and expression of pro-inflammatory response genes CYP1A1, Bax, Bcl2, TNFα, TGFβ, IL-1a, IL-6 and IL-8 (FIG. 7C). For cell viability and cellular GSH, the results are expressed as mean % of control (vehicle) of three replicates and the error bars represent ±S.E.M. For RT-PCR analysis, data are expressed as mean fold induction over control (vehicle); housekeeping genes were analyzed and the most stable genes across all doses were used to generate a normalization factor that all samples were normalized to.

FIG. 8 shows the effect of doxorubicin on cell structure (FIG. 8A); cell viability (FIG. 8B); cellular glutathione (GSH) (FIG. 8C); and expression of pro-inflammatory response genes CYP1A1, Bax, Bcl2, TNFα, TGFβ, IL-1a, IL-6 and IL-8 (FIG. 8D). For cell viability and cellular GSH, the results are expressed as mean % of control (vehicle) of three replicates and the error bars represent ±S.E.M. For RT-PCR analysis, data are expressed as mean fold induction over control (vehicle); housekeeping genes were analyzed and the most stable genes across all doses were used to generate a normalization factor that all samples were normalized to.

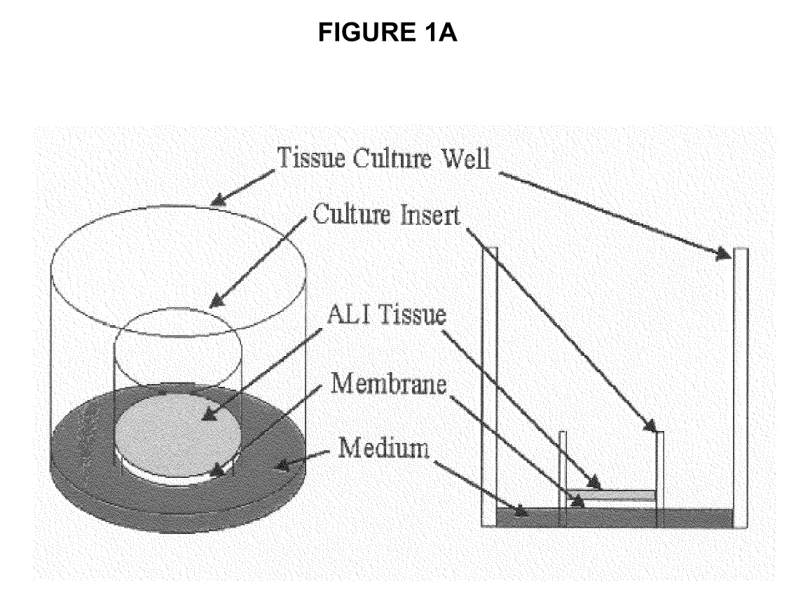

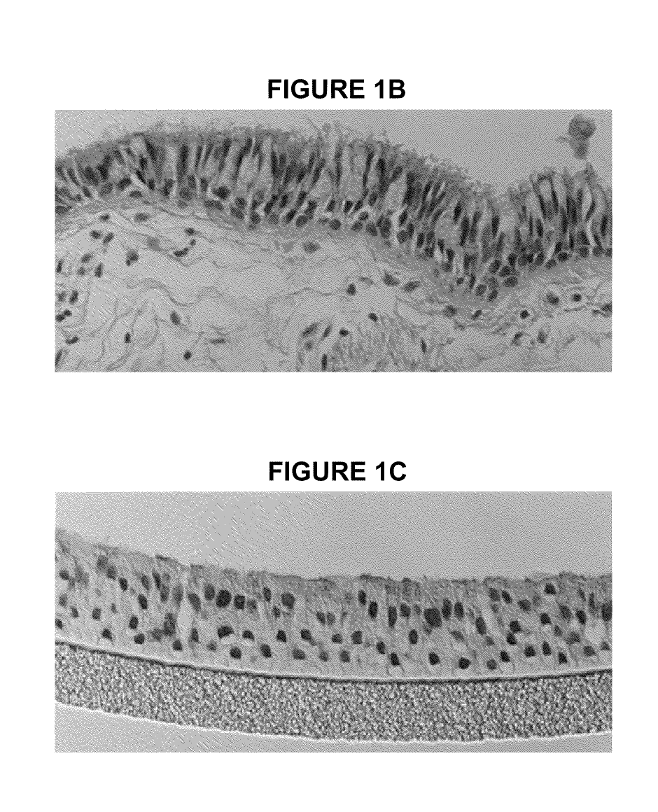

### DETAILED DESCRIPTION OF THE INVENTION

Before explaining various aspects and embodiments of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary and not exhaustive. Also, it is to be understood that the phraseology and terminology employed herein is for the purpose of description and should not be regarded as limiting.

All references cited in this application are expressly incorporated by reference in their entirety.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al., Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following definitions are used throughout the present disclosure: “NOEL” is an abbreviation for No Effect Level, i.e., the highest concentration of the chemical compound at which a measurable toxic effect of the chemical compound is not observable; IC₅₀ is an abbreviation for a measure of the effectiveness of a compound in inhibiting biological or biochemical function, i.e. how much of a particular drug or other substance (inhibitor) is needed to inhibit a given biological process (or component of a process, i.e. an enzyme, cell, cell receptor or microorganism) by half; IC₉₀ is an abbreviation for a measure of the effectiveness of a compound in inhibiting biological or biochemical function, i.e. how much of a particular drug or other substance (inhibitor) is needed to inhibit a given biological process (or component of a process, i.e. an enzyme, cell, cell receptor or microorganism) by 90%.

In a broad sense, the invention relates to improved in vitro methods for detecting and/or analyzing the respiratory toxicity of a substance (e.g., pharmaceutical, cosmetic, biological, or chemical compound or composition). In certain aspects, the invention relates to a method comprising (a) culturing mammalian cells; (b) contacting the mammalian cells with a concentration of the compound; (c) measuring the expression level of one or more marker genes in the mammalian cells after contacting the cells with the compound; (d) monitoring multiple endpoints of cell viability and general cell health; (e) conducting a computational analysis of the concentration of the compound used to contact the cells, and the measured expression level(s) of the one or more marker genes; and (f) determining a predicted in vivo respiratory toxicity value based on the computational analysis.

In certain embodiments of the methods described herein, the mammalian cells are selected from cell types associated with the respiratory system of a mammal, such as synthetic airway in vitro models derived from epithelial cells from, for example, tracheal and/or bronchial tissue (e.g., the 3D EPIAIRWAY model from MatTek, Ashland, Mass.); cells derived from lung tissue including such non-limiting examples as cell lines NCI-H460, NCI-H549, NCI-H661, NCI-H292, BEAS-2B (available from the American Type Culture Collection (ATCC), Manassas, Va.; see also, Franssen-van Hal, N. L. W., et al., Arch Biochem Biophys., (2005), 439:32-41); Clara cells (see, e.g., Massaro et al., Am J Physiol Lung Cell Mol Physiol, (1994); 266:101-106); precision cut tissue slices of lung (see, Vickers & Fisher, Expert Opinion on Drug Metabolism & Toxicology, (December 1995 (online)), 1(4):687-699; and Stefaniak, M. S., et al., In Vitro Toxicology, (1992) 5:(1):7-19). The cells can be derived from any type of mammal. In certain embodiments the cells are human, rat, or mouse cells. In further embodiments, the cells are human cells.

Techniques employed in mammalian primary cell culture and cell line cultures are well known to those of skill in that art. Indeed, in the case of commercially available cell lines, such cell lines are generally sold accompanied by specific directions of growth, media and conditions that are preferred for that given cell line.

Once the cell cultures are thus established, various concentrations of the chemical compound being tested are added to each cell media and the cells are allowed to grow exposed to the various concentrations of a test chemical compound for 6, 24, and 72 hours. Furthermore, the cells may be exposed to the test chemical compound at any given phase in the growth cycle. For example, in some embodiments, it may be desirable to contact the cells with the compound at the same time as a new cell culture is initiated. Alternatively, it may be desirable to add the compound when the cells have reached confluent growth or arc in log growth phase. Determining the particular growth phase that cells are in is achieved through methods well known to those of skill in the art.

The varying concentrations of the given test compound are selected with the goal of including some concentrations at which no toxic effect is observed and also at least two or more higher concentrations at which a toxic effect is observed. A further consideration is to run the assays at concentrations of a compound that can be achieved in vivo. For example, assaying several concentrations within the range from 0 micromolar to about 300 micromolar is commonly useful to achieve these goals. It will be possible or even desirable to conduct certain of these assays at concentrations higher than 300 micromolar, such as, for example, 350 micromolar, 400 micromolar, 450 micromolar, 500 micromolar, 600 micromolar, 700 micromolar, 800 micromolar, 900 micromolar, or even at millimolar concentrations. The estimated therapeutically effective concentration or the estimated maximum workplace exposure concentration of a compound provides initial guidance as to upper ranges of concentrations to test. For certain chemicals, the maximum soluble concentration may be used to establish the highest exposure concentration. An important component of the proposed system is the use of the 3D human respiratory model. These cells are grown at an air-liquid interface, which allows for the application of insoluble test material directly to the test system. Examples include dusts, particles, creams, oils, vapors, liquids, and gases.

In certain embodiments of the assays used in the method of the invention, the test compound concentration range under which the assay is conducted comprises dosing solutions which yield final growth media concentration of 0.05 micromolar, 0.1 micromolar, 1.0 micromolar, 5.0 micromolar, 10.0 micromolar, 20.0 micromolar, 50.0 micromolar, 100 micromolar, 300 micromolar, and up to millimolar concentrations of the compound in culture media. As mentioned, these are exemplary ranges, and it is envisioned that any given assay will be run in at least two different concentrations, and the concentration dosing may comprise, for example, 4, 5, 6, 7, 8, 9,10, 11, 12, 13, 14, 15 or more concentrations of the compound being tested. Such concentrations may yield, for example, a media concentration of 0.05 micromolar, 0.1 micromolar, 0.5 micromolar, 1.0 micromolar, 2.0 micromolar, 3.0 micromolar, 4.0 micromolar, 5.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 55.0 micromolar, 60.0 micromolar, 65.0 micromolar, 70.0 micromolar, 75.0 micromolar, 80.0 micromolar, 85.0 micromolar, 90.0 micromolar, 95.0 micromolar, 80.0 micromolar, 110.0 micromolar, 120.0 micromolar, 130.0 micromolar, 140.0 micromolar, 150.0 micromolar, 160.0 micromolar, 170.0 micromolar, 180.0 micromolar, 190.0 micromolar, 200.0 micromolar, 210.0 micromolar, 220.0 micromolar, 230.0 micromolar, 240.0 micromolar, 250.0 micromolar, 260.0 micromolar, 270.0 micromolar, 280.0 micromolar, 290.0 micromolar, and 300 micromolar, or more (e.g., millimolar concentrations) in culture media. It will be apparent that a cost-benefit balancing exists in which the testing of more concentrations over the desired range provides additional information, but at additional cost, due to the increased number of cell cultures, assay reagents, and time required. In one embodiment, between three to ten different (i.e., three, four, five, six, seven, eight, nine, or ten) concentrations over the range of 0 micromolar to 300 micromolar are screened.

Typically, the various assays described herein can comprise culturing cells seeded in 6, 12, 24, or 96 well plates or 384 cell plates. The cells are then each exposed to the test compounds over a concentration range, for example, 0-300 micromolar. The cells are incubated in these concentrations for a given period of, for example, 6, 24, and 72 hours. In some instances a single time point may be sufficient. In one embodiment, all the assays are performed in at least triplicates at the same time such that a complete set of data are generated under similar conditions of culture, time and handling. However, it may be that the assays are performed in batches within a few days of each other.

In certain embodiments of the methods described herein, the marker genes correlate to cell stress and/or cell toxicity, such as the non-limiting examples of genes associated with a pro-inflammatory response, apoptosis, oxidative stress, and the like. Non-limiting examples of such genes include CYP1A1; Bax; Bcl2; TNFα; TGFβ; IL-1a; IL-6; IL-8; quinone reductase; CD-86; aldo-keto reductase; thioredoxin; and thioredoxin reductase, and the like. Each marker or group of markers is selected based on key biochemical pathways that it represents.

In some embodiments, the methods of the invention comprise at least one marker gene. In other embodiments the methods of the invention comprise more than one marker gene, i.e., two marker genes, three marker genes, four marker genes, five marker genes, six marker genes, seven marker genes, eight marker genes, nine marker genes, ten marker genes, or more than 10 marker genes. Expression levels may be measured by any appropriate method of measuring gene expression, including, but not limited to polymerase chain reaction (PCR), reverse transcriptase PCR (RT-PCR), fluorescent in situ hybridization (FISH), branched DNA (bDNA) assay, differential display, RNA interference, reporter genes, microarrays, and proteomics. For instance, expression levels may be measured by RT-PCR in at least triplicates. The standard protocols for performing RT-PCR may be obtained from Invitrogen, (Carlsbad, Calif.). Standard methods and protocols for measuring gene expression are described in the literature and in treatises such as Sambrook et al., “Molecular Cloning: A Laboratory Manual” and Avison, Matthew, “Measuring Gene Expression,” Taylor and Francis (2006). These protocols are incorporated herein by reference in their entirety.

In certain embodiments of the methods described herein, the multiple endpoints are specific to cell stress and/or cell toxicity. Such endpoints include biochemical and physiological markers that are indicative of cell stress and/or toxicity, including the non-limiting examples of biochemical of physiological assays for detection of cytokine expression, apoptosis (such as caspase 3, 8, 9 activation), cell replication/proliferation, mitochondrial function, oxidative stress (such as mitosox, DCFDA, 8-isoprostane, glutathione (i.e., GSH/GSSG ratios)); membrane leakage markers (such as LDH, adenylate cyclase, and the like), metabolic activation, metabolic stability, enzyme induction, enzyme inhibition, and interaction with cell membrane transporters, and other assays known in the art. The specific assay to monitor any of the given parameters is not considered crucial so long as that assay is considered by those of skill in the art to provide an appropriate indication of the particular biochemical or molecular biological endpoint to be determined, such as information about mitochondrial function, energy balance, membrane integrity, cell replication, and the like.

Compounds that produce direct effects on the cells typically alter mitochondrial function, by either up- or down regulating oxidative respiration. This means that cellular energy in the form of ATP may be altered. Mitochondrial function can be used as an indicator of cytotoxicity and cell proliferation. Healthy mitochondria catalyze the reduction of 3-(4,5-dimethylthiazol-2-yl)-2,5-diphenyltetrazolium bromide (MTT) to a blue or purple formazan compound. The relatively insoluble formazan blue is extracted into isopropanol and the absorbance of the extract measured. A high absorbance value indicates viable cells and functional mitochondria. Conversely, a decrease in the intensity of color suggests either a loss of cells, or direct toxic effects on the mitochondria. The MTT assay is well known to those of skill in the art and has been described in for example, the MTT mitochondrial dye assay is described in Mosmann, J. Immunol. Methods 65, 55-63, 1983 and in Denizot et al., J. Immunol. Methods. 89, 271-277, 1986. A similar assay that monitors XTT mitochondrial dye is described by Roehm et al., J. Immunol. Methods, 142, 257-265, 1991. In addition, those of skill in the art also may determine mitochondrial function by performing for example an Alamar Blue assay [Goegan et al., Toxicol. In vitro 9, 257-266. 1995], a Rhodamine 123 assay, or a cytochrome C oxidase assay.

ATP provides the primary energy source for many cellular processes and is required to sustain cell and tissue viability. Intracellular levels of ATP decrease rapidly during necrosis or apoptosis. Therefore, changes in the cellular concentration of ATP can be used as a general indicator of cell health. When normalized on a per cell basis, ATP can provide information on the energy status of the cell and may provide a marker to assess early changes in glycolytic or mitochondrial function. Assays that allow a determination of ADP/ATP energy balance are well known in the art (Kangas et al., Med. Biol., 62, 338-343, 1984).

Other assays for determining membrane integrity include, but are not limited to, assays that determine lactate dehydrogenase activity, aspartyl aminotransferase, alanine aminotransferase, isocitrate dehydrogenase, sorbitol dehydrogenase, glutamate dehydrogenase, ornithine carbamyl transferase, γ-glutamyl transferase, and alkaline phosphatase.

In various embodiments, the methods can also comprise analysis of the multiple endpoints (genetic markers, biochemical markers, and physiological markers) as a function of the concentration response and time response of the test compound or composition. Taken together, these data provide an in vitro toxicity index which can relate respiratory toxicity to inhaled exposure concentrations. As discussed herein, the methods can utilize a trained model, generated from compounds with known respiratory effect, to categorize the respiratory toxicity profile of a test compound or composition (e.g., non-toxic, severely toxic/corrosive, irritant, and sensitizer).

In various embodiments, the computational analysis comprises determining cell viability at each concentration of the test compound. If at a given exposure concentration the viability is less than 50% then the information is not used and the algorithm moves to the next highest exposure concentration. The data from each endpoint is then evaluated. Endpoints are selected for their specificity to a toxicity category. Concentration response data provides quantitative information. A combination of data that encompass the no effect level (NOEL), IC₅₀, and IC₉₀ points on the concentration response curve provide reference values for estimating the in vivo respiratory toxicity and for categorizing the severity of the chemical response. The data are then binned into responses specific for each toxicity category. The link to in vivo exposure is based on a data set of known lung or respiratory toxicants and known exposure levels and toxicity categories. The in vitro data are then correlated to the in vivo data and are used to develop a relational equation, such as regression analysis that then allows for evaluation of the toxicity of the test compound.

The IC₅₀ values of the test chemicals are related to known inhalation exposure concentrations of known respiratory toxicants. The mean IC₅₀ values of the general cell health endpoints (e.g., membrane integrity, mitochondrial function, cell proliferation) provide data for the y axis or dependant variable while the in vivo inhalation exposures are represented on the x axis as independent variables. A database of known inhalation toxicants is built and maintained that allows for compound comparison based on chemical structure and physical chemical properties such as logP, solubility, vapor pressure, logD and molecular weight.

In one aspect, the invention provides a toxicity index, which provides a lung specificity value. This is calculated by comparing the mean IC₅₀ values of the toxicity endpoints measured in the lung cell model to the mean IC₅₀ values measured in a hepatocyte (liver model). The ratio of lung to liver IC₅₀S provides the toxicity index. If the index is equal to 1.0 there is no preference for lung toxicity. The chemical would be expected to produce toxicity in liver or lung. If the toxicity index is less than one, toxicity is more specific for lung. The smaller the index value, the more specific the toxicity is for lung. If the index is greater than 1.0, then the chemical has a higher specificity for liver. By using the liver cell as the central benchmark it is possible to develop a sense of specificity for the target organ (lung).

In another aspect, the invention provides a method for screening a compound for in vivo respiratory toxicity comprising: (a) culturing mammalian cells; (b) contacting the mammalian cells with a concentration of the compound; (c) measuring the expression level of one or more marker genes in the mammalian cells after contacting the cells with the compound; (d) monitoring multiple endpoints of cell viability and general cell health after contacting the cells with the compound; (e) conducting a computational analysis of the concentration of the compound used to contact the cells, and the measured expression level(s) of the one or more marker genes; (f) determining a predicted in vivo toxicity value based on the computational analysis; and (g) determining whether the toxicity value of the compound falls within acceptable limits for the particular in vivo use.

In an embodiment of this aspect, the determined toxicity value of a compound can be evaluated and determined to be within acceptable limits for particular in vivo applications (e.g., route and frequency of administration, required dosage, etc) by one of skill in the art. Such toxicity values are established and are known by those of skill in the art.

In a further aspect, the invention provides a method for categorizing the in vivo respiratory toxicity of a compound, comprising: (a) culturing mammalian cells; (b) contacting the mammalian cells with a concentration of the compound; (c) measuring the expression level of one or more marker genes in the mammalian cells cells after contacting the cells with the compound; (d) monitoring multiple endpoints of cell viability and general cell health after contacting the cells with the compound; (e) conducting a computational analysis of the concentration of the compound used to contact the cells and the measured expression level(s) of the one or more marker genes; and (f) determining a predicted in vivo toxicity value based on the computational analysis; wherein the computational analysis comprises a comparison of the data from the compound with data gathered from at least two compounds with known respiratory toxicity profiles, wherein the each of the two compounds with known respiratory toxicity profiles are classified independently as a respiratory sensitizer, a respiratory irritant, or a respiratory corrosive, wherein the at least two compounds are not members of the same toxicity profile class.

In embodiments of this aspect the categorizing a compound and/or a composition comprises analyzing data and determining the “respiratory” class of a compound based on the physiological effect the compound(s) exert on an in vitro cell model system as described herein (or as otherwise known in the art). In certain embodiments, the compound or composition is classified as having no respiratory effect (non-toxic), a respiratory sensitizer, a respiratory irritant, or a respiratory corrosive (highly toxic), based on a comparison of the respiratory toxicity profile of the compound or composition to at least one respiratory toxicity profile of at least one compound of known respiratory effect (i.e., no effect/non-toxic, sensitizer, irritant, or corrosive/highly toxic). Non-limiting examples of respiratory sensitizers include gluaraldehyde, phthalic anhydride, piperazine dihydrochloride, cyclohexan-1,2-dicarboxylic anhydride, ethylenediamine, tetrachlorophthalic anhydride, trimellitic anhydride, methyltetrahydrophthalic anhydride, and the like. Non-limiting examples of respiratory irritants include lipopolysaccharide, silica, beryllium salts, platinum salts, chromium salts, and the like. Non-limiting examples of respiratory corrosives include cadmium chloride, bleomycin, paraquat, toluene diisocyanate, arsenic trioxide, warfarin, phosgene, and the like. Chemicals that represent each category are maintained in a database that allows unknown compounds to be compared to well characterized toxicity profiles. Chemicals with similar profiles are in the same toxicity category. Compounds that are classified within the same category demonstrate at least two characteristic endpoint profile values that are sufficiently similar to the endpoint values of each other (e.g., certain marker genes, oxidative stress levels, time/concentration dependence, and the like as described herein). In some embodiments, compounds within the same category demonstrate at least three, four, five, six, seven, eight, nine, or ten or more endpoint values that a sufficiently similar to the endpoint values of each other. In some embodiments, compounds that demonstrate two, three, four, or five or more endpoint values that are not sufficiently similar to each other are classified in different toxicity categories.

In another aspect, the invention provides a kit that comprises from one to all of the necessary components for performing the in vitro methods and assays described herein. In some embodiments of this aspect, all the necessary components for conducting the detection of expression of key genes associated with respiratory sensitization and/or toxicity may be packaged into a kit. In these embodiments, a kit for use in a detection of expression of key genes associated with respiratory sensitization and/or toxicity, as described herein, comprises a packaged set of reagents for conducting the detection by means of RT-PCR. The kits also can comprise the reagents for measuring and analyzing gene expression data that encompass the no effect level (NOEL), IC₅₀, and IC₉₀ points on the concentration response curve. The kits also can comprise other reagents for conducting additional detection and assays.

In addition to the reagents, the kit can also includes instructions packaged with the reagents for performing one or more variations of the detection assay of the invention using the reagents. The instructions may be fixed in any tangible medium, such as printed paper, or a computer readable magnetic or optical medium, or instructions to reference a remote computer data source such as a world wide web page accessible via the Internet

In some embodiments the invention provides a kit for use in a respiratory toxicity assay, the kit comprising a packaged set of reagents for conducting a first cytotoxicity assay selected from the group consisting of a cycle evaluation assay, mitochondrial function assay, energy balance assay and cell death assay; a second cytotoxicity assay selected from the group consisting of a cycle evaluation assay, mitochondrial function assay, energy balance assay and cell death assay; and a third cytotoxicity assay selected from the group consisting of a cycle evaluation assay, mitochondrial function assay, energy balance assay and cell death assay; wherein said first, second and third cytotoxicity assays are distinct from each other. The kits also may comprise the reagents for conducting a fourth or fifth cytotoxicity assay selected from selected from the group consisting of a cycle evaluation assay, mitochondrial function assay, energy balance assay and cell death assay. In addition to the reagents, the kit preferably also includes instructions packaged with the reagents for performing one or more variations of the multiple endpoint assay of the invention using the reagents. The instructions may be fixed in any tangible medium, such as printed paper, or a computer-readable magnetic or optical medium, or instructions to reference a remote computer data source such as a world wide web page accessible via the internet.

While the cells, marker genes and individual endpoints in the present invention have been described previously, the combination of the cell model, the measurement of the expression of one or more marker genes, the monitoring of multiple endpoints and the computational analysis is a novel aspect of the invention.

The invention is further illustrated by the following examples, which should not be construed as limiting in any way. While some embodiments have been illustrated and described, it should be understood that changes and modifications can be made therein in accordance with ordinary skill in the art without departing from the invention in its broader aspects as defined in the following claims.

### Examples

The results presented here validate the method of the invention using a three-dimensional human respiratory cell model (EPIAIRWAY) in which respiratory toxicity could be produced by exposure to classical respiratory toxins bleomycin, cadmium chloride, beryllium sulfate, lipopolysaccharide (LPS) and silica; and a known cardiotoxin, doxorubicin. In vivo studies have shown that bleomycin induces expression of TNFα, TGFβ, IL-1 and IL-6 in the lung epithelia of mice (Cavarra et al., 2004). Cadmium has been shown to induce expression of IL-6 in human lung fibroblast cells (Shin, 1996). Little is known about the effects of doxorubicin on lung epithelia

Cell viability was determined by combining the formation of formazan (MTT) with the histological analysis of the models cell structure. Oxidative stress was analyzed by measuring the depletion of intracellular reduced glutathione. qRT-PCR was used to identify markers for increased xenobiotic metabolism, cytokine production, apoptosis and oxidative stress.

### Methods

### Materials

Dulbecco's Phosphate Buffered Saline (DPBS) may be obtained from MatTek (Ashland, Mass.). Bleomycin is purchased from Toronto Research Chemicals, Inc. (North York, ON Canada). Anhydrous cadmium chloride is purchased from Alfa Aesar (Ward Hill, Mass.). Doxorubicin, beryllium sulfate and lipopolysaccharide (LPS) from E. coli are purchased from Sigma (St. Louis, Mo.), while silica (Min-U-Sil® 5) is obtained from U.S. Silica Co. (Berkeley Springs, W.Va.).

### Cell Culture

The three-dimensional in vitro human EPIAIRWAY system may be purchased from MatTek (Ashland, Mass.). Cultures are maintained with supplied culture media according to the manufacturer's instructions. Briefly, EPIAIRWAY 96-well plates are allowed to equilibrate in 250 μL media for at least 18 hours overnight at 37° C. with 5% CO₂ upon arrival. Media is changed every other day if cells are not used immediately after equilibration. This model contains highly differentiated human tracheal/bronchial epithelial cells cultured at the liquid-air interface, very closely resembling the epithelial tissue of the upper respiratory tract.

### Dosing

In the Examples described below, dosing solutions were prepared in DPBS (MatTek, Ashland, Mass.). The EPIAIRWAY cells were dosed with 100 μL of the following concentrations: 1, 10, 30 and 100 μM bleomycin, cadmium chloride, beryllium sulfate and doxorubicin; 1, 10, 30 and 100 μg/cm² silica; and 1, 10, 30 and 100 ng/mL LPS. Bleomycin has been shown to be relatively non-toxic, yet induce cytokine release in vitro in human lung epithelia and fibroblasts at concentrations of 10 μg/mL (˜7.1 μM) (Sato et al., 1999 and Takamizawa et al., 1999). The dosing regimen used here brackets this dose. Cadmium chloride exhibits cytotoxicity in human epithelial cells at 50 μM and induces significant LDH release in human fetal lung fibroblasts at 8.75 μM, two concentrations well within the dosing range used here (Han et al., 2007 and Yang et al., 1997). Beryllium has been shown to be cytotoxic to fetal human lung cells and human lung fibroblasts at concentrations as low as 5.5 μM (Sakaguchi et al., 1984 and Lehnert et al., 2001). Again, the dosing regimen used brackets this concentration. After a phase 1 study, doxorubicin was identified as safe for inhalation up to 7.5 mg/m² every 3 weeks (Otterson et al., 2007). This calculates to 750 ng/cm². The concentrations and volumes dosed calculate to 483 ng/cm² (1 μM), 4.83 μg/cm² (10 μM), 14.5 μg/cm² (30 μM) and 48.3 μg/cm² (100 μM) doxorubicin, bracketing the safe range. MIN-U-SIL 5 silica, with an average surface area of 5.1 cm²/g gives the following concentrations dosed: 0.051 cm ²/cm² (1 μg/cm²), 0.51 cm²/cm² (10 μg/cm²), 1.53 cm²/cm² (30 μg/cm²) and 5.1 cm²/cm² (100 μg/cm²). The overload dose for fine particle dusts is between 1 and 3 cm²/cm² (Faux et al., 2003). LPS is commonly dosed in concentrations ranging from 0.1 ng/mL to 10 μg/mL (Pugin et al. 1993). In order to analyze the acute response of EPIAIRWAY cells to LPS, cells are exposed to LPS concentrations ranging from 1 ng/mL to 100 ng/mL. Following the exposure period, the dosing solutions are removed and cells are analyzed for changes in structure, cell viability, oxidative stress and gene expression.

### Histology

Cells were isolated using a 3 mm biopsy punch (Miltex, York, Pa.) and immediately placed in PROTOCOL 10% Neutral Buffered Formalin (Fisher Diagnostics, Middletown, Va.). Histology was performed essentially as described by Sheehan and Hrapchak (1980). Briefly, cells were processed and embedded into paraffin wax blocks. The blocks were sectioned at 3 μm, adhered to slides and stained with hematoxylin and eosin. Photos were taken at 10× magnification.

### Cytotoxicity (MTT Assay)

Cell viability was determined by measuring the reduction of 3-[4,5- dimethylthiazol-2-yl]2,5-diphenyltetrazolium bromide (MTT). The cells in each well were evaluated for their ability to reduce soluble-MTT (yellow) to formazan-MTT (purple). An MTT stock solution was prepared at in complete medium just prior to use and warmed to 37° C. in a water bath. Once the media and dosing solution was removed from all wells, MTT solution was added to the basal side of each well and the plate was allowed to incubate at 37° C. for 3-4 hr. Media was removed and the purple formazan product was extracted using anhydrous isopropanol applied to both the apical and basal side of the cells. Sample absorbance was read at 570 nm and reference absorbance at 650 nm with a Packard Fusion or equivalent plate reader.

### Intracellular Glutathione (GSH) Levels

Intracellular glutathione levels were determined essentially as described by Griffith (1980) with modifications. At the end of the exposure period, the medium was removed from the cells and metaphosphoric acid (MPA) was added to each well. Plates were then shaken for 5 min at room temperature and stored at −20° C. until needed.

Isolation of Total RNA and qRT-PCR Analysis

Total RNA was isolated from EPIAIRWAY human respiratory cells by the TRIZOL (Invitrogen, Carlsbad, Calif.) method (Chomczynski and Sacchi, 1987)with modifications. Briefly, cells were punched out using a biopsy punch (Miltex, York, Pa.) and immediately placed in TRIZOL. The samples were then frozen at −80° C. until needed. After thawing, the isolation procedure followed the manufacturer's instructions.

Induction of target gene mRNA was determined as follows, by standard one-step qRT-PCR techniques using multiple internal control genes for normalization with a few modifications (Vandesompele et al, 2002). RT-PCR was performed using the Quantitect® SYBR Green RT-PCR kit (Qiagen, Valencia, Calif.) according to the manufacturer's instructions for 20 μL reactions. All primers were QUANTITECHT Primer Assay (Qiagen, Valencia, Calif.) containing forward and reverse primers in the same tube (see Table 1, below). Briefly, 50 ng of RNA in 20 μL reactions are set up in 384-well RT-PCR plates (Roche Diagnostics Corp., Indianapolis, Ind.) and placed in a LIGHTCYCLER 480 real time PCR machine (Roche Diagnostics Corp., Indianapolis, Ind.). One-step PCR was set as follows; reverse transcription was 1 cycle of 50° C. for 30 minutes followed by 95° C. for 15 minutes. Amplification was set for 45 cycles of 94° C. for 15 seconds, 55° C. for 20 seconds and 72° C. for 20 seconds. Melting curve was generated by 1 cycle of 95° C. for 5 seconds followed by 65° C. for 1 minute. Data were analyzed using the LIGHTCYCLER 480 software version 1.5.0.39 (Roche Diagnostics Corp., Indianapolis, Ind.). The expression levels of multiple housekeeping genes were determined and an average fold induction over vehicle was elucidated for each housekeeping gene. The three housekeeping genes with the least variability across all doses and compounds tested were averaged to generate a normalization factor. Samples were normalized to control and then to the normalization factor to obtain a fold induction vs. control.

TABLE 1

| GENE | CATALOG NO. | ACCESSION NO. | AMPLICON SIZE |
|---|---|---|---|
| GAPDH | QT00079247 | NM_002046 | 95 bp |
| 18S | QT00199367 | X03205 | 149 bp |
| B2M | QT00088935 | NM_004048 | 98 bp |
| ACTB | QT01680476 | NM_001101 | 104 bp |
| CYP1A1 | QT00012341 | NM_000499 | 127 bp |
| Bax | QT00031192 | NM_004324 | 111 bp |
| Bcl2 | QT00025011 | NM_000633 | 80 bp |
| TNF-α | QT01079561 | NM_000594 | 104 bp |
| TGF-β | QT00000728 | NM_000660 | 108 bp |
| IL-1α | QT00001127 | NM_000575 | 74 bp |
| IL-6 | QT00083720 | NM_000600 | 107 bp |
| IL-8 | QT00000322 | NM_000584 | 102 bp |

### Example 1

### Bleomycin

Bleomycin is a glycosylated peptide antibiotic originally isolated from the fungus Streptomyces verticillus. It is commonly used to treat many types of cancer and is well known for its induction of a potentially fatal respiratory condition called bleomycin-induced pneumonitis or BIP. Bleomycin is known to induce IL-1, IL-6, IL-8, TNF-α and TGF-β expression in the lungs of mice in vivo (Cavarra et al., 2004, Chaudhary et al., 2006, Piguet et al., 1989, Zhang et al., 1997, Karmiol et al., 1993, Micallef et al., 1992, and Phan and Kunkel, 1992). Bleomycin also induces oxidative stress in the pulmonary epithelia of mice, which is known to be a crucial component of its toxicity (Manoury et al., 2005). Extended exposure of the human lung cells to bleomycin can lead to activation of fibroblasts via cytokine signaling, which may eventually lead to fibrosis (Moseley et al., 1986, Sugerman et al., 1985 and Schmidt et al., 1982). Although the exact mechanism of in vivo cytotoxicity in humans remains unclear, the effects appears to be similar to those observed in mice: damage due to free radicals and uncontrolled release of bleomycin-induced cytokines Bleomycin has been shown in humans to induce expression of IL-1 and IL-18 in vivo (Hoshino et al., 2009), and IL-1 and TNF-α in human alveolar macrophages in culture (Scheule et al., 1992). Additionally, IL-8 has been shown to be induced by bleomycin treatment in human microvascular pulmonary endothelial cells (Fichtner et al., 2004).

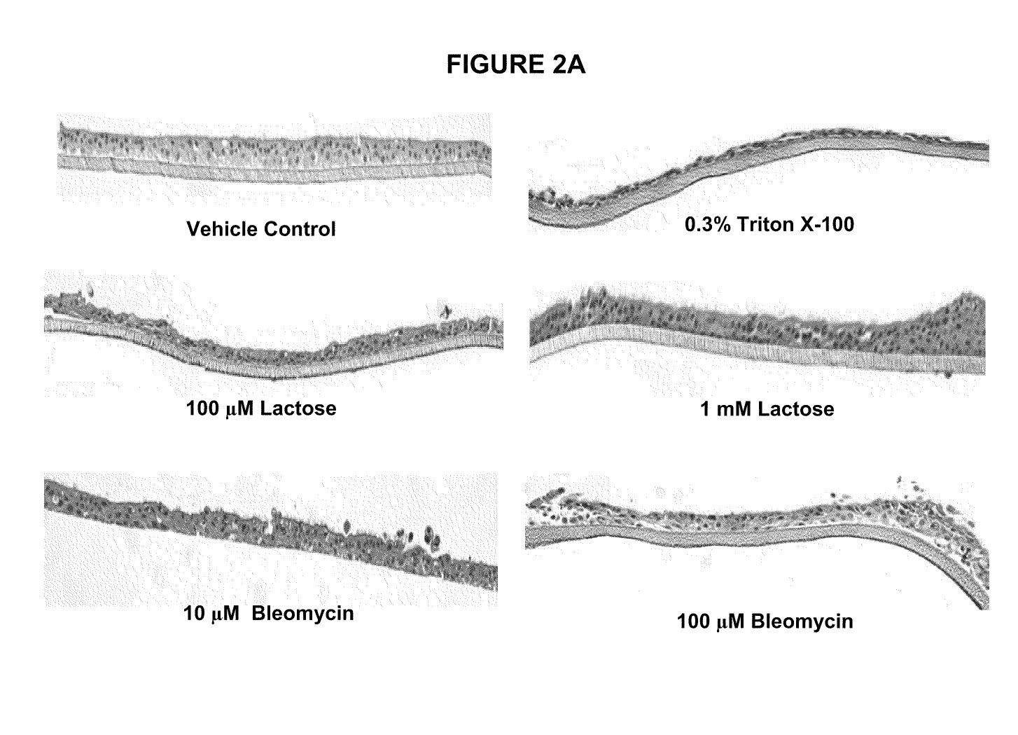

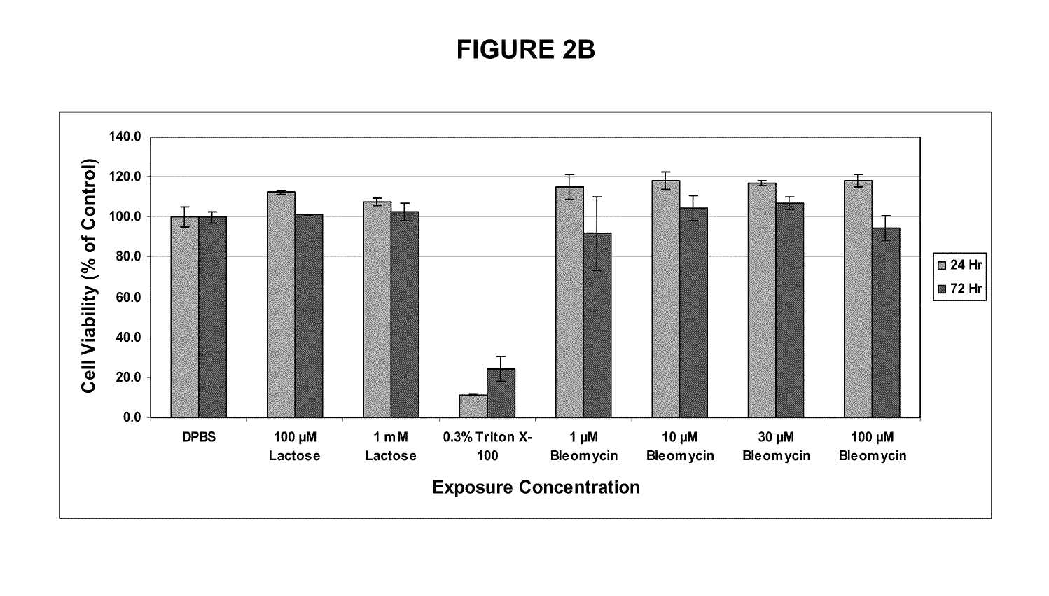

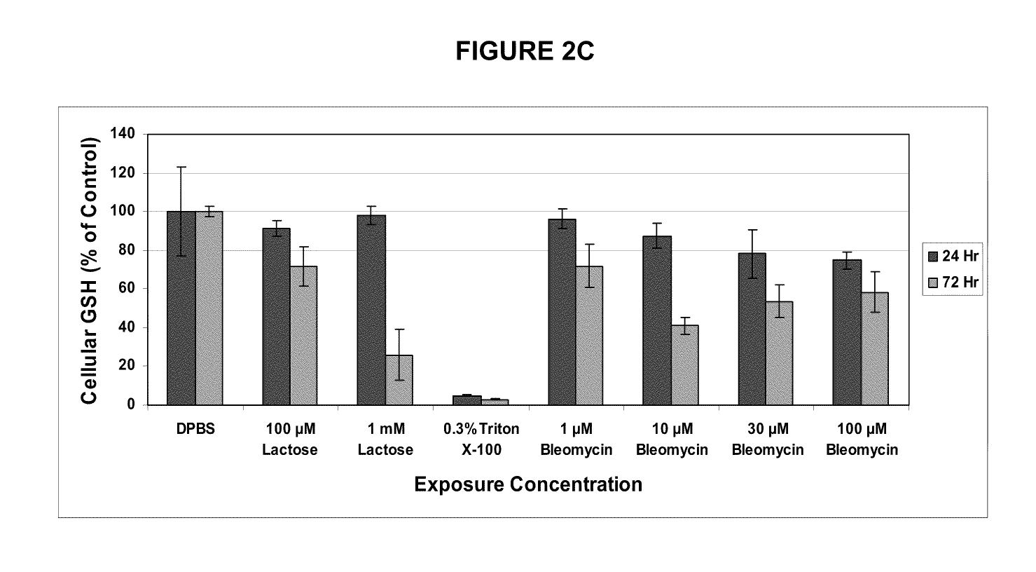

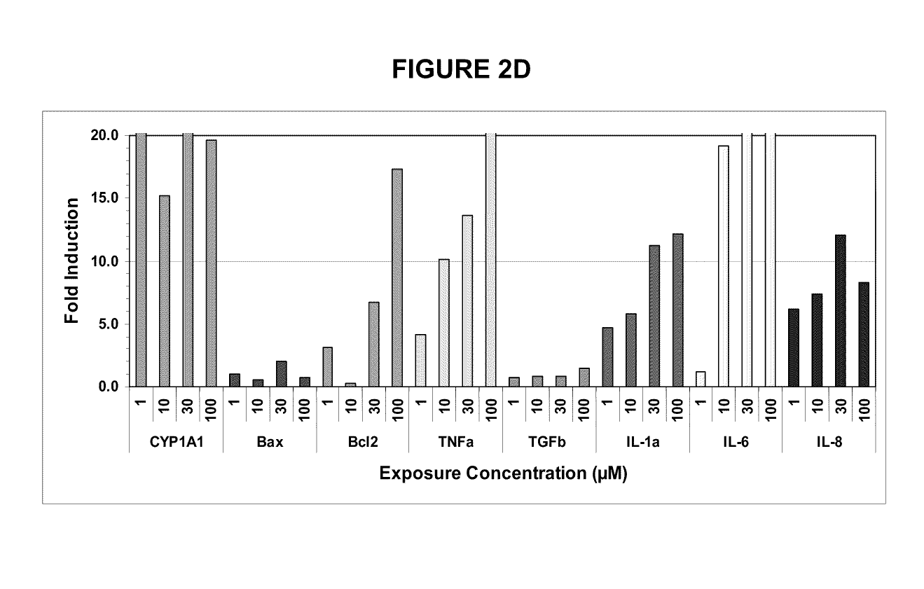

To assess cell viability and structural integrity, cells were treated on the apical surface with 1, 10, 30 and 100 μM bleomycin. Cells were exposed for 24 and 72 hours. Cell viability was assessed by MTT at 24 and 72 hours (FIG. 2B), while the structural integrity of the EPIAIRWAY 3-D model system was assessed by histology at 24 hours (FIG. 2A). Bleomycin reduced cell viability only to 90% of control after 72 hour exposure, with no effect on viability after 24 hours. Interestingly, histology showed significant structural breakdown and loss of cells at 24 hours (FIG. 2A).

To analyze oxidative stress, the EPIAIRWAY 3-D model system was exposed on the apical surface with 1, 10, 30 and 100 μM bleomycin for 24 and 72 hours. Total cellular GSH levels were analyzed in order to assess oxidative stress. Bleomycin is known to induce oxidative stress in lung epithelia and showed a dose dependant decrease in total cellular GSH to 75% of control with 100 μM after 24 hours (FIG. 2C). Exposure to bleomycin for 72 hours showed a reduction in cellular GSH to 71% of control at 1 μM and between 41% and 58% of control for 10, 30 and 100 μM.

Real time RT-PCR was used to determine the expression of genes involved in metabolic activity, oxidative stress, apoptosis and pro-inflammatory biomarkers in response to exposure of EPIAIRWAY cells. Cells were exposed on the apical surface to 1, 10, 30 and 100 μM bleomycin. A change in the relative abundance of the target gene mRNA>2-fold was considered to be significant. As seen in FIG. 2D, bleomycin greatly induced expression of CYP1A1, in a dose-independent manner. The proinflammatory markers, TNF-α, IL-1α, IL-6 and IL-8, were all induced in a dose-dependent manner, with TNF-α and IL-6 reaching more than a 20-fold increase over control tissues. The anti-apoptotic gene Bcl2 was also induced.

The results suggest that the mechanism of bleomycin toxicity in human lung tissue is similar to the mechanism in mice whereby bleomycin induces oxidative stress and release of pro-inflammatory cytokines leading to acute lung injury. Intracellular GSH levels show that bleomycin induced oxidative stress after 24 hours of exposure. In addition, the results demonstrate that bleomycin induces expression of CYP1A1, Bcl2, TNF-α, IL-1α, IL-6 and IL-8. These results correlate very well with known in vivo responses. In fact, all the data, with the exception of TGF-β expression, correlate well with in vivo results. TGF-β has been shown to be induced in vivo in response to bleomycin in mice (Phan and Kunkel, 1992). But it was not surprising that this induction was not seen after 24 hours of exposure. TGF-β, along with IL-4 and IL-13, are primarily associated with induction of pulmonary fibrosis, which typically is caused by a prolonged exposure to toxic compounds (Giri et al., 1993).

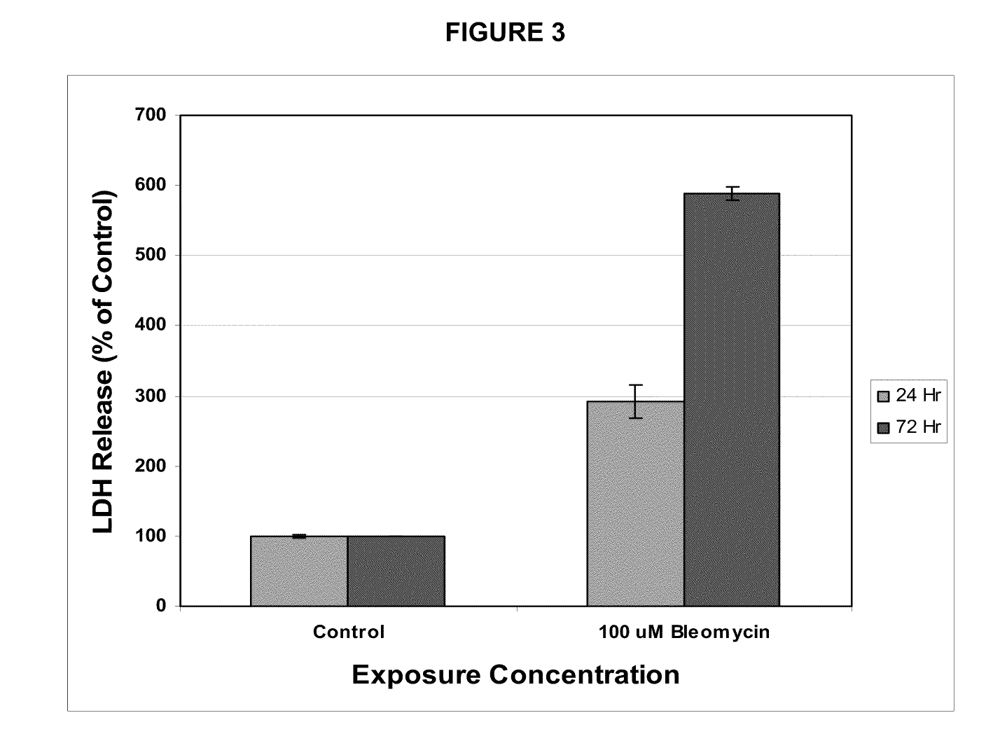

The primary shortcoming of this model system appears to be that the MTT results to not compare well with histology. This may be due to the fact that the basal cell layers have the highest metabolic activity given that they are still rapidly dividing. The compounds may not be able to come into contact and exert its toxic effects on these bottom layers of highly active cells and therefore, they are still able to rapidly reduce the MTT to formazan. In fact, even 0.3% Triton X-100 was unable to kill these bottom layers of cells by 24 hours as seen in the histology. Fortunately, there appears to be a much more concise method to assess cytotoxicity in this model system. Lactate dehydrogenase is a cytosolic enzyme commonly used as a marker for cell membrane permeability, and therefore, cytotoxicity (Davies et al., 1973). As FIG. 3 shows, LDH release into the media after exposure of EPIAIRWAY cells to 100 μM bleomycin is increased 292% when compared to control at 24 hours, and 588% when compared to control by 72 hours. These data confirm the cytotoxicity of bleomycin to the EPIAIRWAY cells and appear to be a much more sensitive marker for cytotoxicity in this model.

### Example 2

### Cadmium Chloride

Although the element cadmium is readily used in the manufacture of batteries, its use in other industries (solder, plastics coatings, metal electroplating etc.) has readily decreased over the years due to its toxicity. Cadmium is known to have many serious effects on health with a half life of 15-20 years once ingested (Jarup et al., 1998 and Jin et al., 1998). Today, the primary route of cadmium exposure is through cigarette smoking (Nandi et al., 1969, Martin et al., 2009), and cadmium is well known for its toxic effects on the lung tissue (Patwardhan et al., 1975, Han et al., 2007 and Kundu et al., 2007). In rats, administration of cadmium induced pulmonary inflammation and induced expression and subsequent increase in circulating levels of IL-6 and TNF-α (Kataranovski et al., 1998). Cadmium exposure has also been shown to induce pulmonary fibrosis with TGF-β co-administration in rats and mice (Lin et al., 1998 and Kasper et al., 2004). In humans, the mechanism of cadmium induced toxicity is not well established.

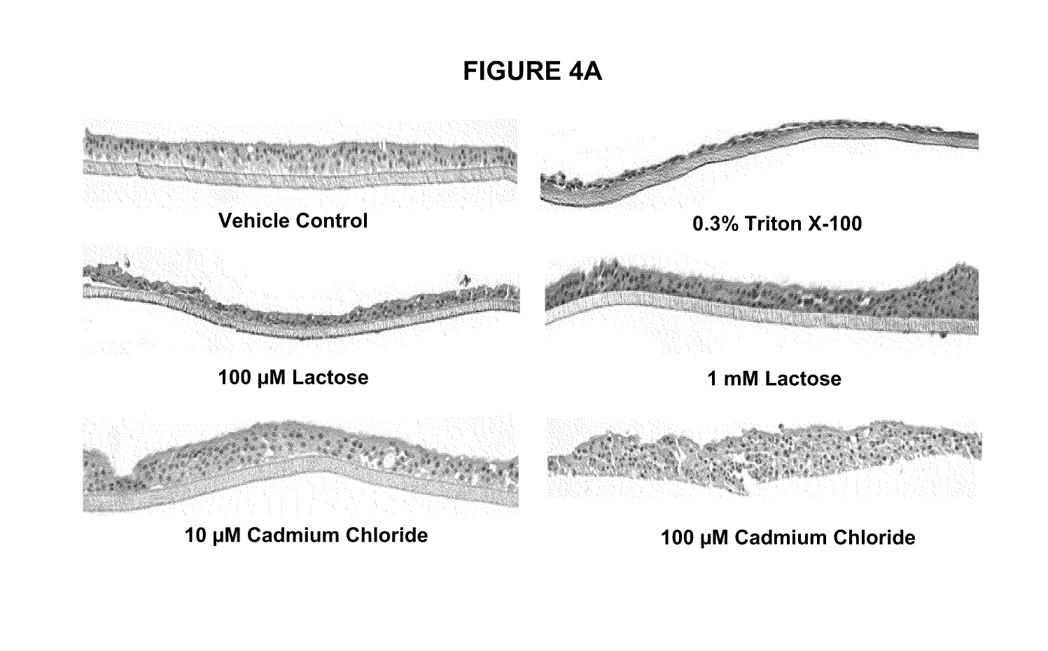

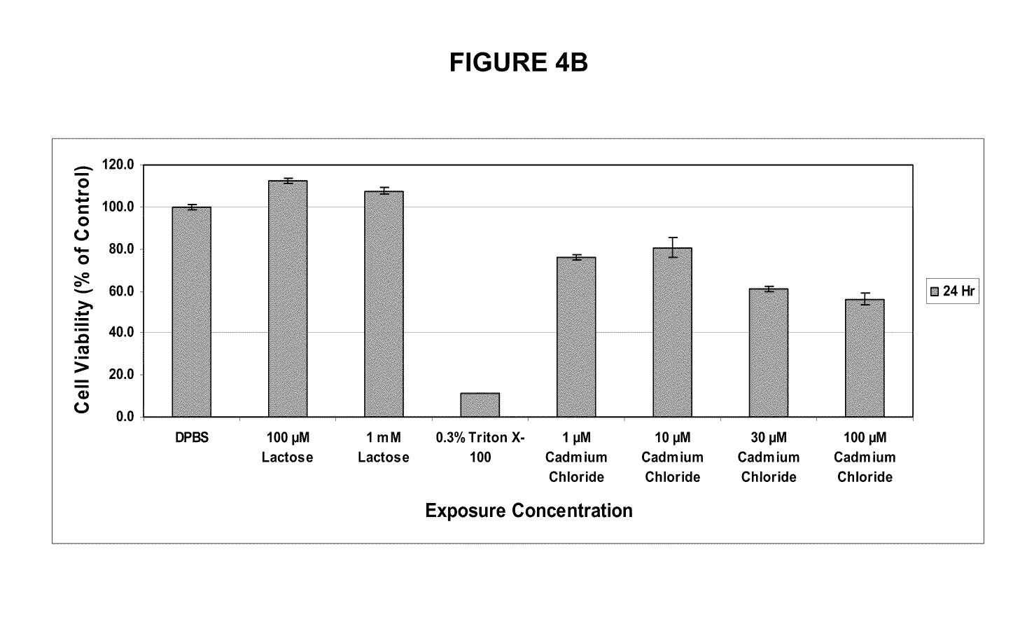

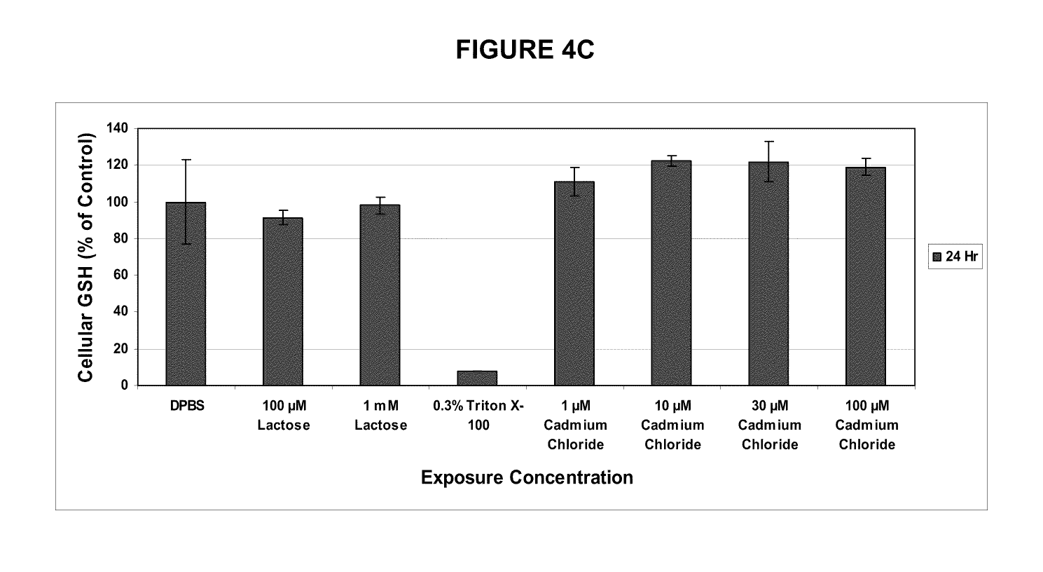

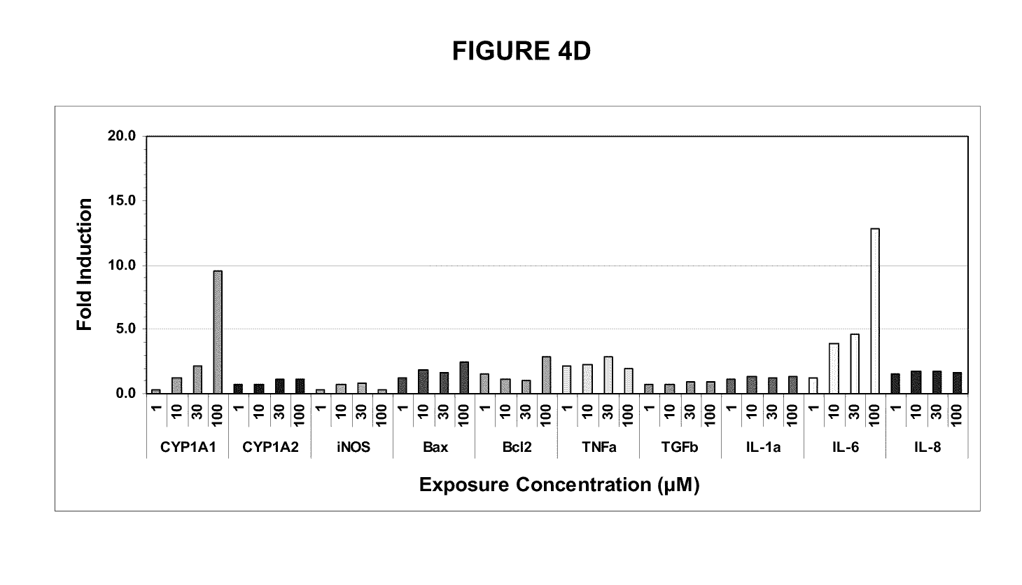

To assess cell viability and structural integrity, cells were treated on the apical surface with 1, 10, 30 and 100 μM cadmium chloride. Cells were exposed for 24 and 72 hours. Cell viability was assessed by MTT at 24 and 72 hours (FIG. 4B), while the structural integrity of the EPIAIRWAY 3-D model system was assessed by histology at 24 hours (FIG. 4A). Cadmium chloride showed a dose dependant reduction in cell viability, down to 56% of control with 100 μM, after 24 hours of exposure. Histology showed cell loss at both 10 μM and 100 μM cadmium chloride concentrations after 24 hour exposure, but with a much more noticeable a breakdown of cellular structure with 100 μM.

To analyze oxidative stress, the EPIAIRWAY 3-D model system was exposed on the apical surface with 1, 10, 30 and 100 μM cadmium chloride for 24 and 72 hours. Total cellular GSH levels were analyzed in order to assess oxidative stress. Cadmium chloride showed no reduction in intracellular GSH levels by 24 hours (FIG. 4C).

Real time RT-PCR was used to determine the expression of genes involved in metabolic activity, oxidative stress, apoptosis and pro-inflammatory biomarkers in response to exposure of EPIAIRWAY cells. Cells were exposed on the apical surface to 1, 10, 30 and 100 μM cadmium chloride. A change in the relative abundance of the target gene mRNA>2-fold was considered to be significant. As seen in FIG. 4D, cadmium chloride substantially induced expression of CYP1A1 and IL-6 in a dose dependant manner, and a mild induction of TNF-α was also observed, however this induction was not dose-dependant. The apoptosis markers Bax and Bcl2 were both mildly induced with 100 μM cadmium chloride in EPIAIRWAY cells.

Thus, cadmium chloride has no effect on intracellular GSH levels after 24 hours of exposure in our model. This suggests that cadmium chloride did not induce oxidative stress, though oxidative stress is thought to be significant to in vivo respiratory toxicity and carcinogenesis (Joseph, 2009). Also, cadmium chloride induced expression of CYP1A1 in EPIAIRWAY cells. Cadmium chloride is known to induce expression and activity of CYP1A1 in human liver cells (Elbekai and El-Kadi, 2007). Cigarette smoke, containing significant amounts of cadmium, is also known to induce CYP1A1 expression in human lung cells (McLemore et al., 1990). It is quite possible, based on these findings, that cadmium is a major contributor to the increased expression of CYP1A1 in human lung cells in response to cigarette smoke. The results also show that cadmium chloride mildly induces expression of the pro-inflammatory cytokine TNF-α, and substantially induces expression of IL-6. These data correlate well with the in vivo data observed by Shin (1996), who showed IL-6 is upregulated in the lungs of humans exposed to cadmium. In mice, TNF-α has been shown to be upregulated upon systemic exposure to cadmium, but this is, to our knowledge, the first report that cadmium directly induces the expression of TNF-α in human lung cells. Bax and Bcl2 were both mildly upregulated at 24 hours with exposure to 100 μM cadmium chloride, perhaps suggesting the cytotoxicity is due to necrotic, not apoptotic, cell death.

### Example 3

### Beryllium

Beryllium has considerable value in modern industry, with uses in aerospace and nuclear power industries. It is a common component in automobiles, computers and other electronics. Inhaled beryllium particles can cause chemical pneumonitis (Eisenbud et al., 1948), also called acute beryllium pneumonitis. As long as patients avoid further exposure to beryllium, they usually recover, although some cases can progress to chronic beryllium disease or CBD (Sprince et al., 1976). Fortunately, standard practices put into place by the Atomic Energy Commission in 1949 (Eisenbud, 1982) have greatly reduced beryllium exposure. Since acute beryllium disease has virtually disappeared due to these measures, research has focused on the immunology of CBD. Dobis et al. analyzed patients with either CBD or beryllium sensitization and concluded that beryllium can mediate a thiol imbalance leading to oxidative stress which may play a role in the pathology of the disease. However, this work was performed in Ficoll-Hypaque-isolated peripheral blood mononuclear cells, so it is inconclusive as to whether beryllium induces oxidative stress in the lung.

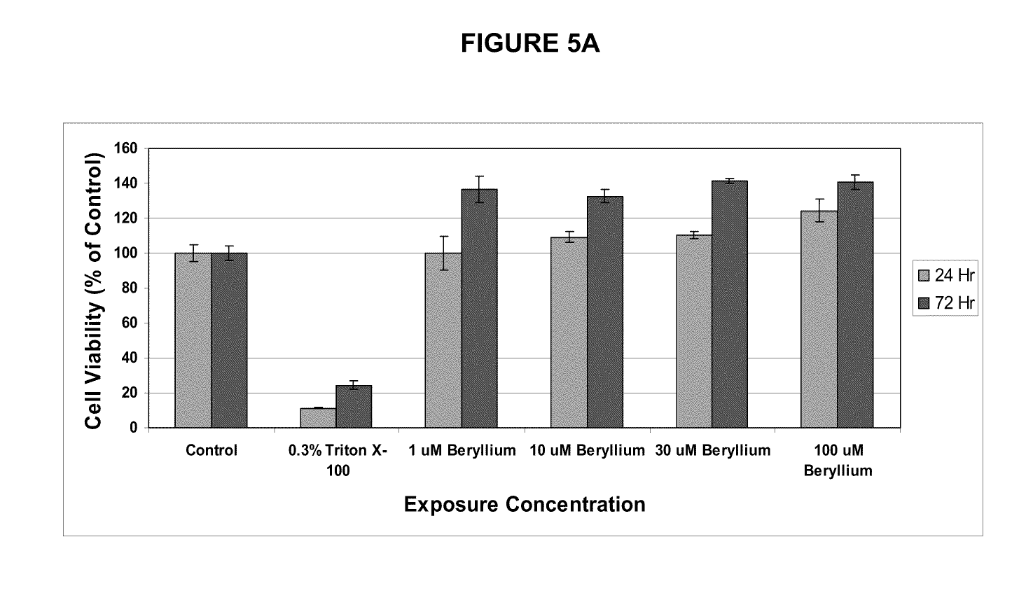

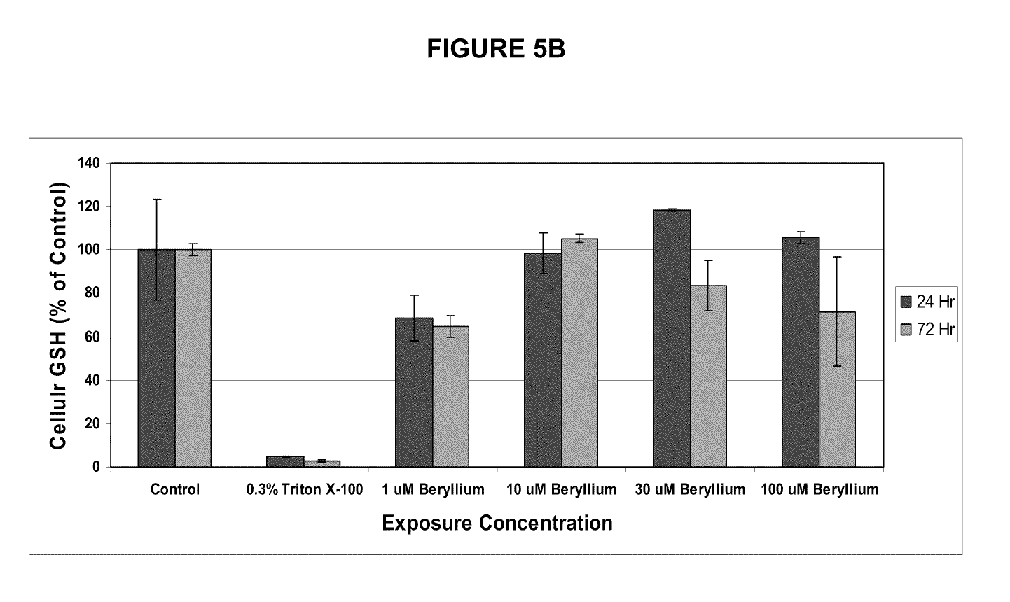

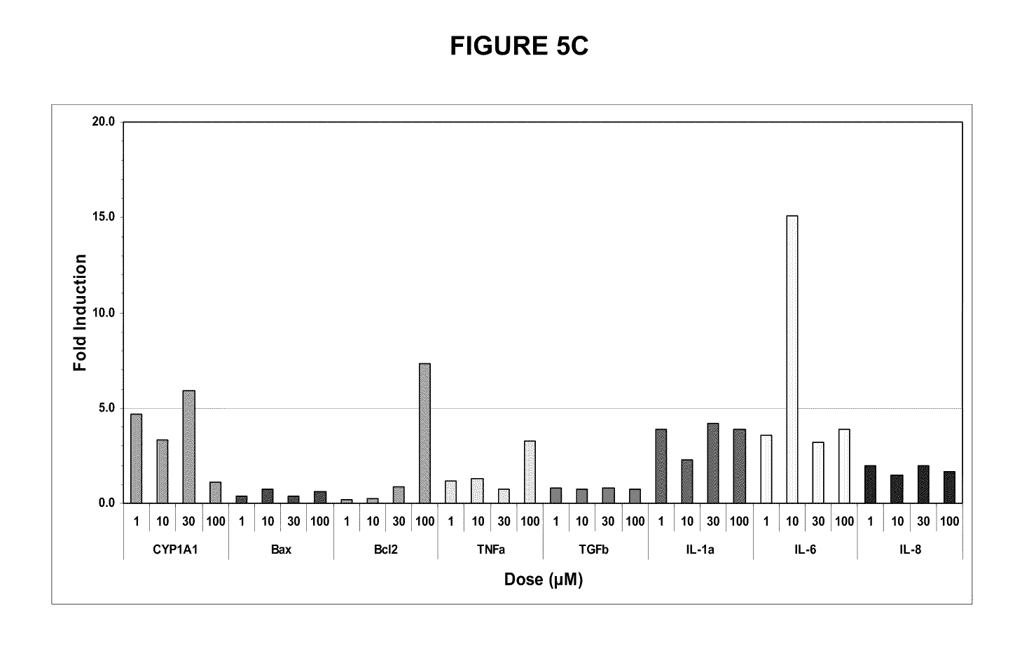

To assess cell viability, cells were treated on the apical surface with 1, 10, 30 and 100 μM beryllium sulfate. Cells were exposed for 24 and 72 hours. Cell viability was assessed by MTT assay at 24 and 72 hours. Beryllium sulfate did not reduce cell viability at any dose, at either time point (FIG. 5A).

To analyze oxidative stress, the EPIAIRWAY 3-D model system was exposed on the apical surface with 1, 10, 30 and 100 μM beryllium sulfate for 24 and 72 hours. Total cellular GSH levels were analyzed in order to assess oxidative stress. Beryllium sulfate mildly reduced intracellular GSH levels after 72 hours of exposure (FIG. 5B). Interestingly, 1 μM beryllium sulfate had the greatest effect, reducing intracellular GSH levels to 65% of control while 30 μM and 100 μM reduced intracellular GSH levels to 83% and 71% of control, respectively. 10 μM beryllium sulfate did not have an effect.

Real time RT-PCR was used to determine the expression of genes involved in metabolic activity, oxidative stress, apoptosis and pro-inflammatory biomarkers in response to exposure of EPIAIRWAY cells. Cells were exposed on the apical surface to 1, 10, 30 and 100 μM beryllium sulfate for 24 hours. A change in the relative abundance of the target gene mRNA>2-fold was considered to be significant. Beryllium sulfate substantially induced CYP1A1, IL-1α and IL-6 expression (FIG. 5C). Also, beryllium sulfate mildly induced the expression of IL-8 at 1 and 30 μM, and TNFα and Bcl2 at 100 μM.

Thus, no reduction in cell viability and no effect of beryllium sulfate on intracellular GSH levels was seen until 72 hours of exposure, at which time intracellular GSH was reduced to 65% of control. It is possible that beryllium induces a mild oxidative stress response or that the oxidative stress is a secondary effect of beryllium exposure. Analysis of bronchoalveolar lavage fluid (BALF) of patients with CBD was shown to have significantly increased levels of TNFα and IL-6 mRNA (Bost et al., 1994, Tinkle et al., 1996 and Tinkle and Newman, 1997). Here we show a mild induction of TNFα with 100 μM beryllium sulfate, and a substantial induction of and IL-6 with all concentrations tested. Previous work has ruled out beryllium induced expression of IL-1β (Bost et al., 1994) and IL-1α was never analyzed. This is due in part to the role of IL-1β in many chronic fibrotic lung diseases and these studies were performed in individuals with CBD. However, we are analyzing cytokine production in response to acute exposure. It is possible the substantially increased expression of IL-1α that we observe in our model is relevant in vivo as well, and during the progression of the disease over time, the expression of IL-1α is downregulated.

### Example 4

### Silica (SiO₂)

Silica (SiO₂), one of the most abundant minerals in the earths crust, has long been known as a respiratory toxin, with prolonged exposure leading to pulmonary fibrosis and cancer. Pulmonary exposure to silica has been reported in the construction, mining, sand blasting, pottery/clay, foundries, sand/gravel, filtration, polishing/grinding and agriculture industries (Donaldson and Borm, 1998). Prolonged exposure to silica has been associated with the development of severe respiratory disorders including silicosis (Davis, 1986), autoimmune diseases (Cooper et al., 2002) and some rare forms of lung cancer (Peretz et al., 2006). Silica induces oxidative stress once it enters the human lung, and there are two major sources of free radical production; particle derived reactive oxygen species (ROS), produced when silica is in an aqueous environment, and cell-derived reactive oxygen species produced by cells in response to silica (Fubini and Hubbard, 2003).

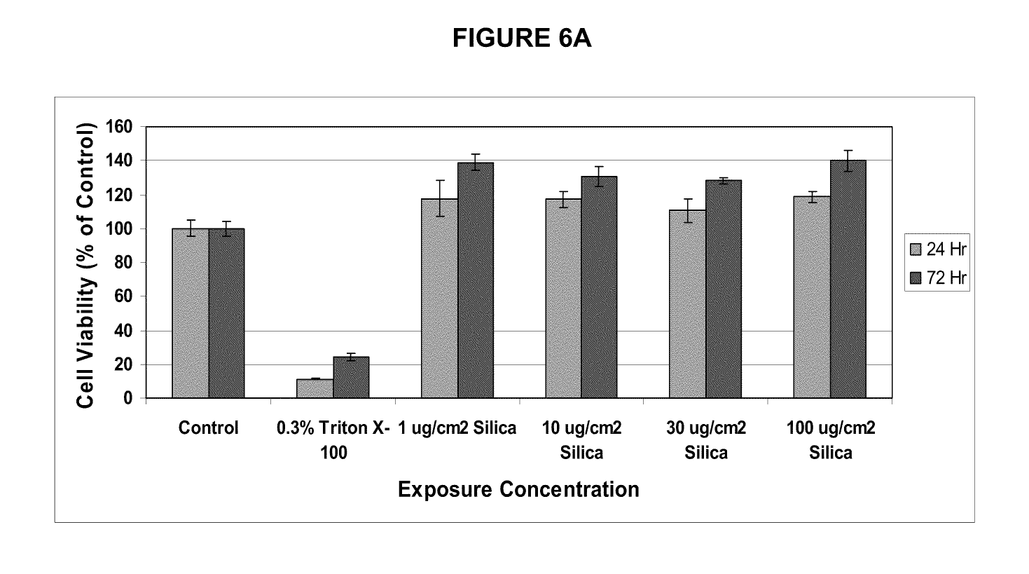

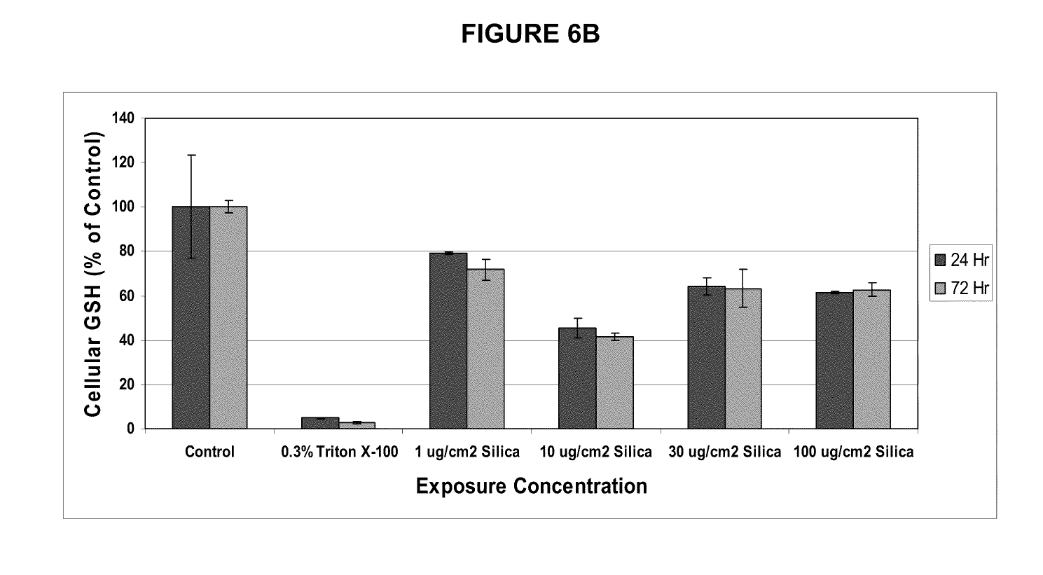

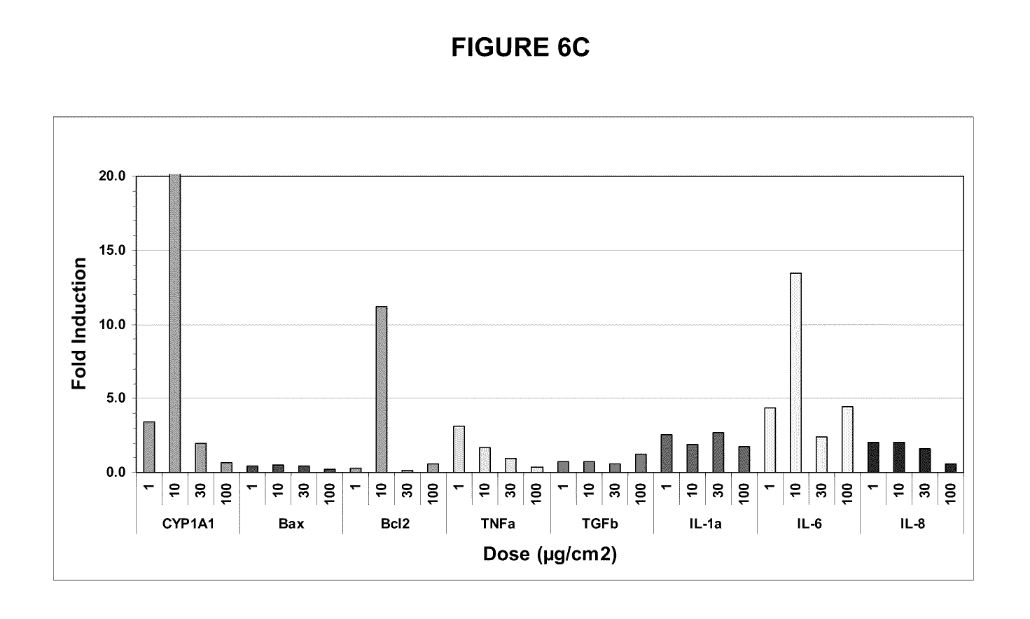

To assess cell viability, cells were treated on the apical surface with 1, 10, 30 and 100 μg/cm² silica. Cell viability was assessed by MTT assay at 24 and 72 hours. Silica did not reduce cell viability at any dose, at either time point (FIG. 6A).

To analyze oxidative stress, the EPIAIRWAY 3-D model system was exposed on the apical surface with 1, 10, 30 and 100 μg/cm² silica. Total cellular GSH levels were analyzed in order to assess oxidative stress. Silica reduced intracellular GSH levels by 24 hours, with the maximum effect, reduced to 45% of control, observed with 10 μg/cm² (FIG. 6B). 1 μg/cm², 30 μg/cm2 and 100 μg/cm² silica reduced intracellular GSH to 79%, 64% and 61% of control, respectively. Interestingly, this reduction remained fairly constant even after 72 hours of exposure.

Real time RT-PCR was used to determine the expression of genes involved in metabolic activity, oxidative stress, apoptosis and pro-inflammatory biomarkers in response to exposure of EPIAIRWAY cells. Cells were exposed on the apical surface to 1, 10, 30 and 100 μg/cm² silica for 24 hours. A change in the relative abundance of the target gene mRNA>2-fold was considered to be significant. Silica substantially induced expression of CYP1A1 and IL-6 while IL-1α and IL-8 were mildly induced (FIG. 6C). In addition, Bcl2 and TNFα were induced but only at one concentration dosed, 10 μM and 1 μM, respectively.

Thus, the in vitro data correlate with known in vivo responses in that silica induces oxidative stress in the EPIAIRWAY model. Intracellular GSH levels were substantially reduced at all doses tested after exposure to silica. Most notably, exposure to 10 μg/cm² intracellular GSH was reduced to 45% of control after 24 hour exposure and to 41% after 72 hours of exposure. There was no reduction in cell viability according to the MTT results across all concentrations and all time points. In addition, a mild induction in expression of the pro-inflammatory cytokine TNFα was seen after 24 hours of exposure with 1 μg/cm². A mild induction of IL-1α and IL-8 were also observed, and a substantial induction of IL-6 was seen across all doses. In vivo, silica exposure to the human lung is known to induce TNFα and IL-6 (Vanhée et al., 1995) and silica is known to induce IL-8 (Herseth et al., 2008) and IL-1α (Baroni et al., 2001) in human cells in vitro. These data correlate well with the known pro-inflammatory response seen in humans. Further, a substantial increase in CYP1A1 levels was seen in the EPIAIRWAY model in response to silica. In rats, pulmonary exposure to silica in vivo and in vitro is known to induce CYP1A1 levels (Becker et al., 2006). It is quite possible that a similar mechanism exists in human pulmonary epithelia.

### Example 5

### Lipopolysaccharides (LPS)

Lipopolysaccharides (LPS) are large molecules in the outer membrane of Gram-negative bacteria and elicit a strong innate immune response via toll-like receptors in diverse eukaryotic species, from insects to humans (reviewed by Imler and Zheng, 2004). LPS is a classical respiratory toxin and the mechanism of pulmonary toxicity has been studied for years (Snell, 1966; Helander et al., 1982; Rylander et al., 1975; and Snella, 1981). Endotoxin released from the bacterial cell wall is considered to be important in triggering the intrapulmonary production of various proinflammatory mediators leading to the induction of reactive oxygen species (ROS) in rats (Goraca and Skibska, 2008), mice (Rocksen et al., 2003) and humans (reviewed in Kollef and Schuster, 1995).

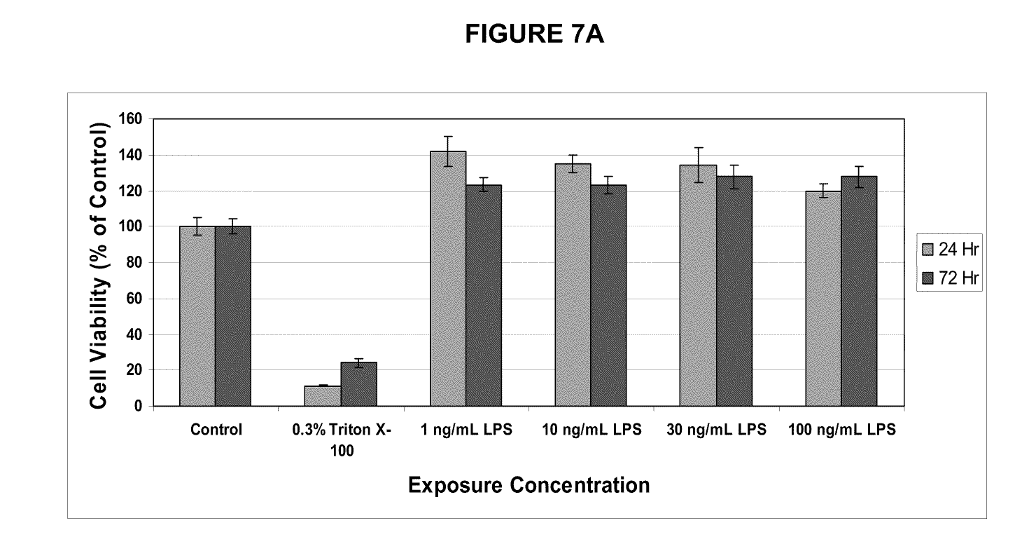

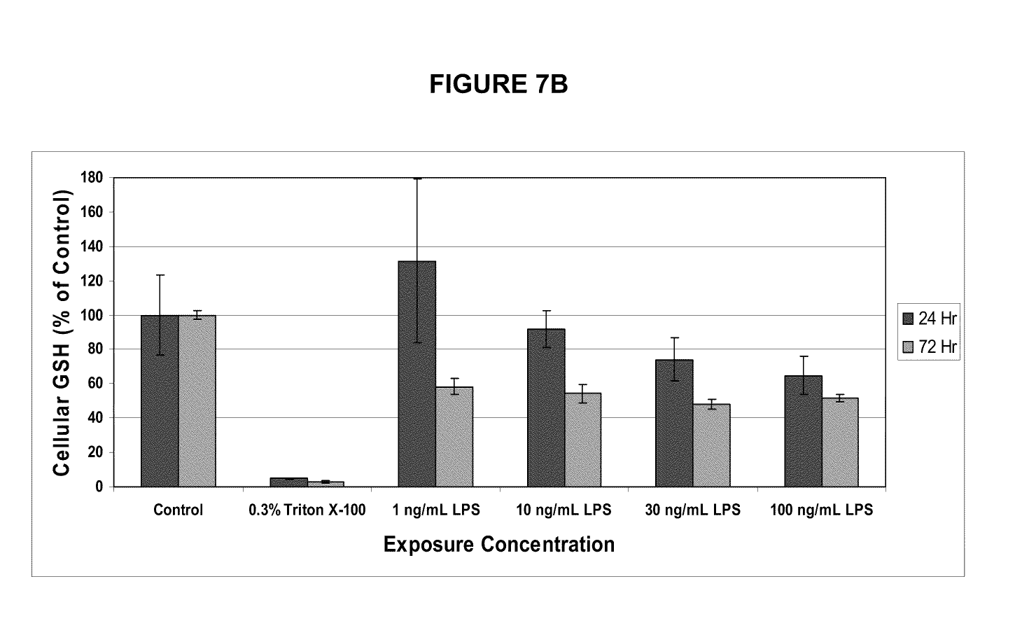

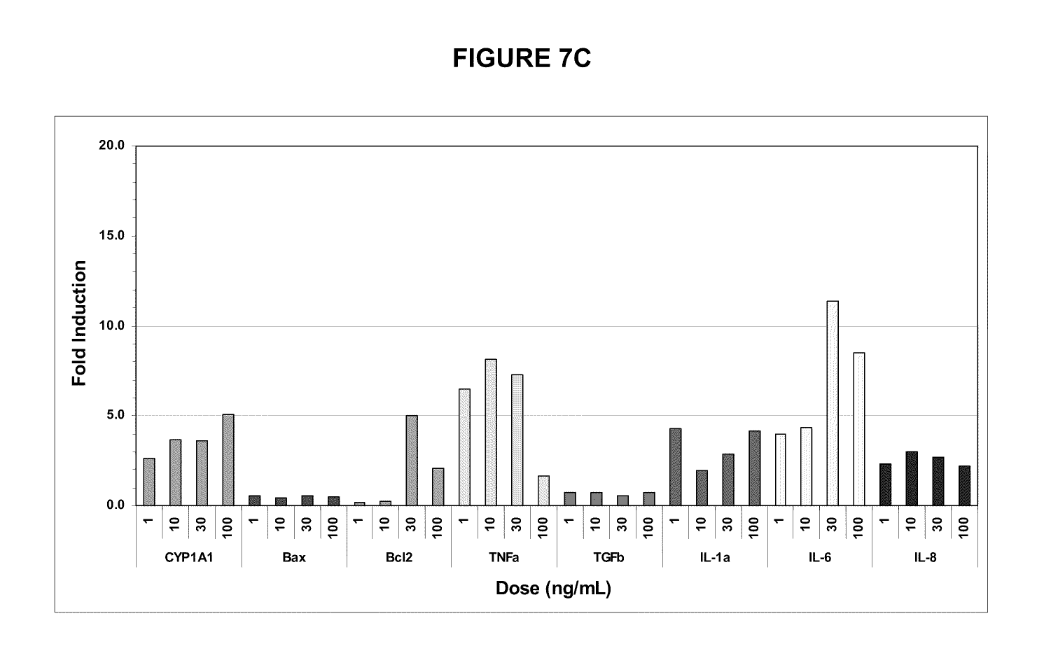

To assess cell viability, cells were treated on the apical surface with 1, 10, 30 and 100 ng/mL LPS. Cells were exposed for 24 and 72 hours. Cell viability was assessed by MTT at 24 and 72 hours. LPS did not reduce cell viability at any dose, at either time point (FIG. 7A).

To analyze oxidative stress, the EPIAIRWAY 3-D model system was exposed on the apical surface with 1, 10, 30 and 100 ng/mL LPS for 24 and 72 hours. Total cellular GSH levels were analyzed in order to assess oxidative stress. LPS reduced intracellular GSH in a dose-dependant manner after 24 hours exposure to 65% of control with 100 ng/mL (FIG. 7B). After 72 hours exposure to LPS, all doses decreased intracellular GSH to between 48% and 58% of control.

Real time RT-PCR was used to determine the expression of genes involved in metabolic activity, oxidative stress, apoptosis and pro-inflammatory biomarkers in response to exposure of EPIAIRWAY cells. Cells were exposed on the apical surface to 1, 10, 30 and 100 ng/mL LPS for 24 hours. A change in the relative abundance of the target gene mRNA>2-fold was considered to be significant. Exposure of EPIAIRWAY cells LPS for 24 hours caused a substantial, dose-independant induction in expression of TNF-α, IL-1α and IL-6 (FIG. 7C). A mild induction of IL-8 is observed with every exposure concentration. Also observed was a dose-dependant increase of CYP1A1 mRNA. Bcl2 expression was also induced, but only with exposure to 30 and 100 ng/mL LPS.

Thus, LPS reduced intracellular GSH levels in a dose dependant manner to 65% of control after 24 hours of exposure. After 72 hours of exposure, the intracellular GSH levels were between 48% and 58% of control. We observed no decrease in cell viability with any concentration at either time point. LPS is also known to induce expression of pro-inflammatory cytokines in lung tissue as well. For example, in mice, intratracheal instillation of LPS results in a significant increase of TNFα, IL-1α, and IL-6 in vivo (Johnston et al, 1998). In BEAS-2B human lung epithelial cells, exposure to LPS is known to induce IL-6 and IL-8 after 10 hours of exposure (Ovrevik et al., 2009) while LPS induces TNFα, IL-6 and IL-8 in ex vivo human lung tissue (Hackett et al., 2008). The induction of TNFα, IL-1α, IL-6 and IL-8 is also known to occur in human lung tissue in vivo upon inhalation of LPS (Hoogerwerf et al., 2008) and injection of LPS. Again, our data corroborate with these data. Here, we show that exposure of EPIAIRWAY cells to LPS for 24 hours induces expression of TNFα, IL-1α, IL-6 and IL-8, correlating with the known in vivo human responses. We also see an increase in expression of Bcl2, possibly as a survival mechanism, and increase in expression of CYP1A1. Systemic LPS exposure is known to decrease the expression and activity of CYP1A1 in the liver of rats (Ke et al., 2001) rabbits (Saitoh et al., 1999) and pigs (Monshouwer et al., 1996). There is evidence, however, that in the rat brain, regulation of CYP1A1 expression in response to LPS is varied (Renton et al., 1999). Therefore, there appears to be tissue-specific modulation of CPY1A1 expression in response to LPS.

### Example 6

### Doxorubicin

Doxorubicin is an anthracycline antibiotic commonly used in conjunction with other anticancer compounds to treat many types of cancer. Doxorubicin is typically administered intravenously, however, given the effectiveness of doxorubicin against many types of lung cancer, it has been suggested that direct exposure of the lungs to doxorubicin may increase its effectiveness (Gagnadoux et al., 2008). It has been shown to be toxic to the lung tissue of dogs (Minchin et al., 1988) and humans (Baciewicz et al., 1991) via lung perfusion but the mechanism of toxicity was not evaluated in either case. Here we set out to determine if doxorubicin should be considered a respiratory toxin and if so, what the mechanism of toxicity is. Doxorubicin is well known for its cardiotoxicity and induction of oxidative stress (Tan et al., 1967, Keizer et al., 1990 and Minotti et al., 2004).

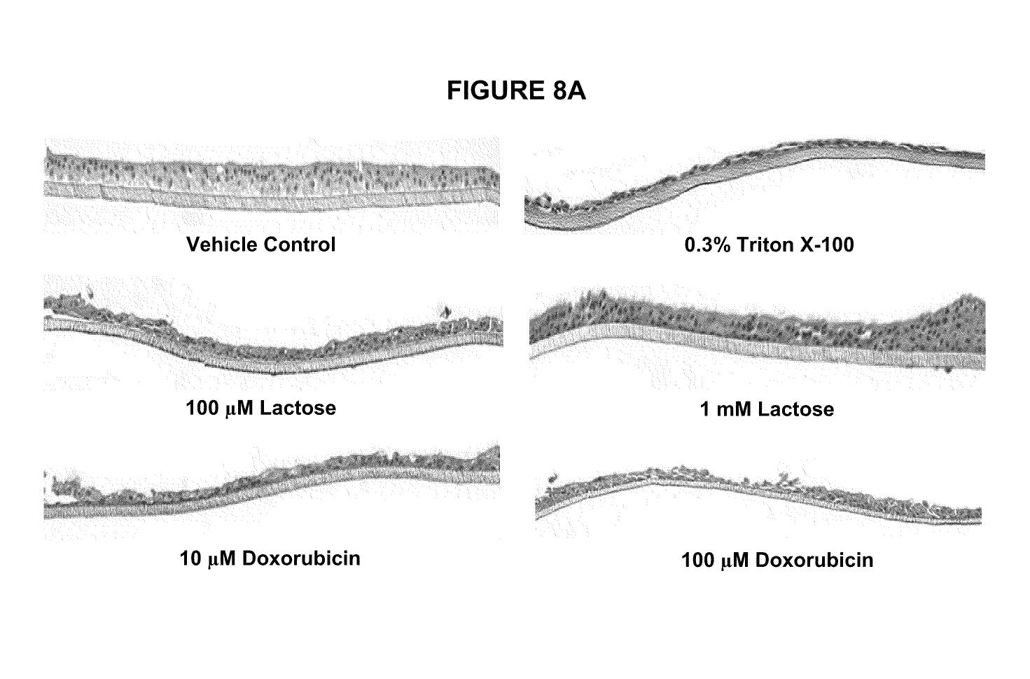

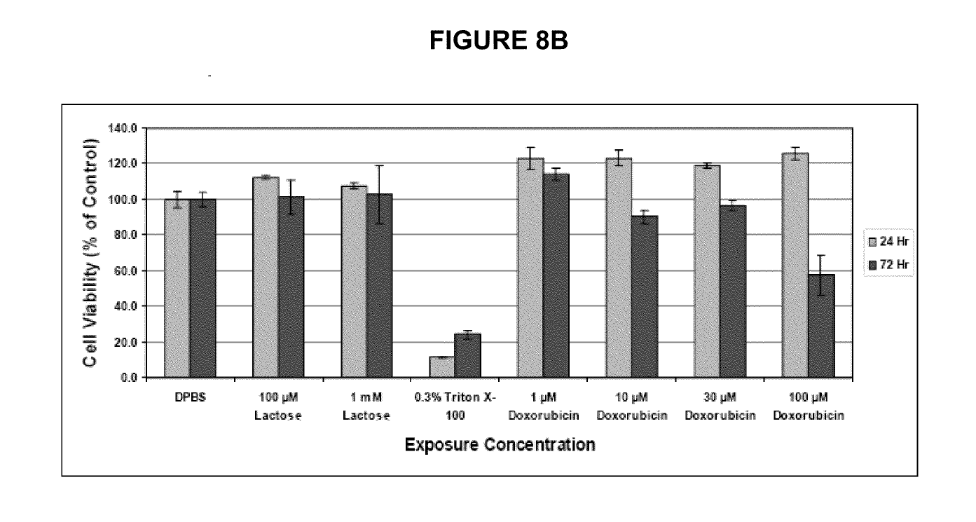

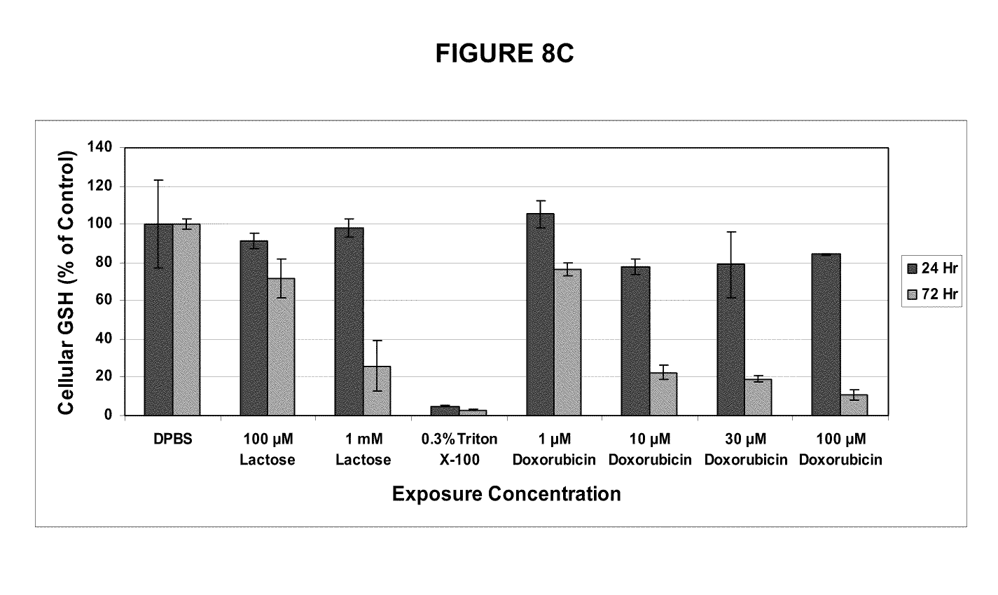

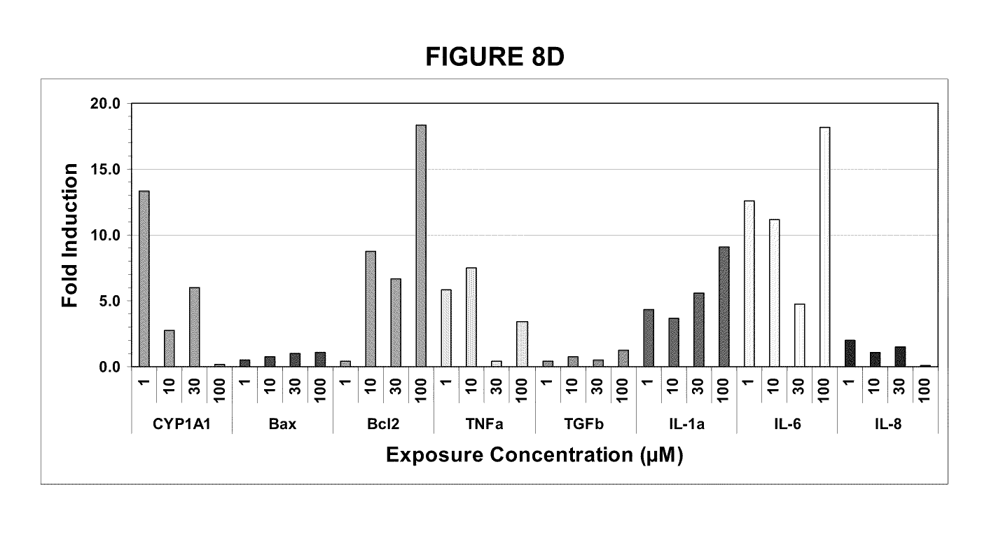

To assess cell viability and structural integrity, cells were treated on the apical surface with 1, 10, 30 and 100 μM doxorubicin. Cells were exposed for 24 and 72 hours. Cell viability was assessed by MTT at 24 and 72 hours (FIG. 8B), while the structural integrity of the EPIAIRWAY 3-D model system was assessed by histology at 24 hours (FIG. 8A). Doxorubicin reduced cell viability to 57% of control with 100 μM, but only after 72 hours. Reduction in cell viability was not observed at 24 hours with any concentration, and 1 μM doxorubicin had no effect on cell viability at either time point. However, similar to bleomycin, histological analysis showed significant structural breakdown and loss of cells at 24 hours (FIG. 8A).

To analyze oxidative stress, the EPIAIRWAY 3-D model system was exposed on the apical surface with 1, 10, 30 and 100 μM doxorubicin for 24 and 72 hours. Total cellular GSH levels were analyzed in order to assess oxidative stress. Doxorubicin, which is known to induce oxidative stress, was shown to reduce intracellular GSH levels to 80% of control at 24 hours with 10, 30 and 100 μM, however at 72 hours of exposure, doxorubicin exhibited a dose dependant effect with a reduction in intracellular GSH levels to 10% of control levels with 100 μM (FIG. 8C).

Real time RT-PCR was used to determine the expression of genes involved in metabolic activity, oxidative stress, apoptosis and pro-inflammatory biomarkers in response to exposure of EPIAIRWAY cells. Cells were exposed on the apical surface to 1, 10, 30 and 100 μM doxorubicin for 24 hours. A change in the relative abundance of the target gene mRNA>2-fold was considered to be significant. Doxorubicin exposure for 24 hours substantially induced expression of CYP1A1, Bcl2, TNFα, IL-1α and IL-6 (FIG. 8D). IL-8 was also mildly induced, but only upon exposure to 1 μM doxorubicin.

Intracellular GSH levels dropped to 80% of control after 24 hours of exposure to 10, 30 and 100 μM doxorubicin and dropped even dramatically to 10% of control after 72 hours of exposure to 100 μM doxorubicin. In addition, doxorubicin induced expression of CYP1A1 in EPIAIRWAY cells. Doxorubicin is known to be highly toxic as well as to induce CYP1A1 in heart cells (Zordoky and El-Kadi, 2008). It may activate CYP1A1 in human respiratory epithelia in the same manner. Doxorubicin also induced expression of Bcl2, TNF-α, IL-1α and IL-6 in this model system, possibly suggesting acute lung injury. Moreover, it is interesting that the ratio of Bax:Bcl2 expression is decreased with bleomycin and doxorubicin treatments. This appears to suggest that at 24 hours, the cells are not undergoing apoptosis and may be increasing Bcl2 expression in response to these compounds in an attempt to survive.

In summary, only two of the tested compounds exhibited reduced cell viability vs. control with MTT: cadmium chloride (56% viable at 24 hours) and doxorubicin (57% viable at 72 hours). However, histological analysis showed cadmium chloride, doxorubicin and bleomycin induced significant structural breakdown and cell loss after 24 hours of exposure. Further, total intracellular GSH levels were 45-80% of control after 24 hours with all compounds tested with the exception of cadmium chloride which showed no reduction of cellular GSH. In addition, the expression of markers for apoptosis (Bax and Bcl2), xenobiotic metabolism (CYP1A1) and the pro-inflammatory response (TNFα, TGFβ, IL-1α, IL-6 and IL-8) were analyzed by qRT-PCR.

The foregoing results demonstrate that the methods of the invention comprising a mammalian cell culture system combined with multiple endpoint analysis provides in vitro toxicity data consistent with the data observed in vivo. The results suggest that an in vitro method can be used to correctly identify known respiratory toxins (bleomycin, cadmium chloride, LPS, silica and beryllium) and accurately characterize and predict the in vivo respiratory toxicity of a compound (e.g., doxorubicin). The data exemplifies the improvement of the method through the use of monitoring multiple endpoints (gene expression, cell morphology, cell viability, and oxidative stress) in combination with the in vitro airway model. As the data indicate, MTT does not appear to be a reliable assay to determine cytotoxicity in this model system for these particular compounds, and that other cellular assays (e.g., cellular ATP levels or LDH release) may better predict respiratory toxicity. The in vitro gene expression profiles very closely resemble the known in vivo responses (see, e.g., Cavarra et al., 2004 and Shin, 1996). Thus, methods for detecting and/or predicting in vivo respiratory toxicity of a compound, where the method comprises multiple endpoints that monitor cell health, oxidative stress and gene expression, provide an improvement over known predictive methods and can provide a better model for characterizing the respiratory toxicity profile of a compound and/or composition.

### REFERENCES

The following references are hereby incorporated by reference in their entirety:

Baciewicz Jr, F. A., Arredondo, M., Chaudhuri, B., Crist, K. A., Basilius, D., Bandyopadhyah, S., Thomford, N. R. and Chaudhuri, P. K. (1991) Pharmacokinetics and toxicity of isolated perfusion of lung with doxorubicin. J. Surg. Res. 50(2):124-128.

Baroni, T., Bodo, M., D'Alessandro A., Conte, C., Calvitti, M, Muzi, G., Lumare, A., Bellocchio, S. and Abbritti, G. (2001) Silica and its antagonistic effects on transforming growth factor-beta in lung fibroblast extracellular matrix production. J. Investig. Med. 49(2):146-156.

Becker, A., Albrecht, C., Knaapen, A. M., Schins, R. P. F., Höhr, D., Ledermann, K. and Borm, P. J. A. (2006) Induction of CYP1A1 in rat lung cells following in vivo and in vitro exposure to quartz. Arch. Toxicol. 80(5):258-268.

Blank, F., Rothen-Rutishauser, B. M., Schurch, S. and Gehr, P. (2006) An optimized in vitro model of the respiratort tract wall to study particle cell interactions. J. Aerosol Med. 19(3):392-405.

Bost, T. W., Riche,s D. W., Schumacher, B., Carré, P. C., Khan, T. Z., Martinez, J. A. and Newman, L. S. (1994) Alveolar macrophages from patients with beryllium disease and sarcoidosis express increased levels of mRNA for tumor necrosis factor-alpha and interleukin-6 but not interleukin-1 beta. Am. J. Respir. Cell. Mol. Biol. 10(5):506-513.

Chomczynski, P. and Sacchi, N. (1987) Single-step method of RNA isolation by acid guanidinium thiocyanate-phenol-chloroform extraction. Anal. Biochem. 162(1): 156-159.

Cavarra, E., Cararro, F., Fineschi, S., Naldini, A., Bartalesi, B., Pucci, A. and Lungarella, G. (2004) Early response to bleomycin is characterized by different cytokine and cytokine receptor profiles in lungs. Am. J. Physiol. Lung Cell Mol. Physiol. 287(6):L1186-1192.

Chaudhary, N. I., Schnapp, A., Park, J. E. (2006) Pharmacologic differentiation of inflammation and fibrosis in the rat bleomycin model. Am. J. Respir. Crit. Care Med. 173(7):769-776.

Cooper, G. S., Miller, F. W. and Germolec, D. R. (2002) Occupational exposures and autoimmune diseases. Int. Immunopharmacol. 2:303-313.

Davies, P., Allison, A. C., Fox, R. I., Polyzonis, M. and Haswell, A. D. (1973) The exocytosis of polymorphonuclear-leukocyte lysosomal enzymes induced by cytochlasin B. Biochem. J. 128(3):78P-79P.

Davis, G. S. (1986) The pathogenesis of silicosis. State of the art Chest. 89:166S-169S.

Donaldson, K. and Borm, P. J. A. (1998) The Quartz Hazard:A variable entity. Ann. Occup. Hyg. 42(5):287-294.

Eisenbud M. (1982) Origins of the standards for control of beryllium disease (1947-1949). Environ. Res. 27:79-88.

Eisenbud, M., Berghout, C. F. and Steadman, L. T. (1948) Environmental studies in plants and laboratories using beryllium: the acute disease. J. Ind. Hyg. 30:281-285.

Elbekai, R. H. and El-Kadi, A. O. (2007) Transcriptional activation and posttranscriptional modification of Cyp 1 al by arsenite, cadmium, and chromium. Toxicol. Lett. 172(3):106-119.

Faux, S. P., Tran, C. L., Miller, B. G., Jones, A. D., Monteiller, C. and Donaldson, K. (2003) In Vitro Determinants of Particulate Toxicity: The dose-metric for poorly soluble dusts. Reasearch Report 154. Norwich, Great Britain: Institute of Occupational medicine for the Health and Safety Executive.

Fichtner, F., Koslowski, R., Augstein, A., Hempel, U., Röhlecke, C., Kasper, M. (2004) Bleomycin induces IL-8 and ICAM-1 expression in microvascular pulmonary endothelial cells. Exp. Toxicol. Pathol. 55(6):497-503.

Fiegel, J., Ehrhardt, C, Schaefer, U. F., Lehr, C. M. and Hanes J. (2003) Large Porous Particle Impingement on Lung Epithelial Cell Monolayers—Toward Improved Particle Characterization in the Lung. Pharma. Res. 20(5): 788-796.

Fubini, B. and Hubbard, A. (2003) Reactive oxygen species (ROS) and reactive nitrogen species (RNS) generation by silica in inflammation and fibrosis. Free Radic. Biol. Med. 34:1507-1516.

Gagnadoux, F., Hureaux, J., Vecellio, L., Urban, T., Le Pape, A., Valo, I., Montharu, J., Leblond, V., Boisdron-Celle, M., Lerondel, S., Majoral, C., Diot, P., Racineux, J. L. and Lemarie, E. (2008) Aerosolized chemotherapy. J. Aerosol Med. Pulm. Drug Deliv. 21(1):61-70.

Giri, S. N., Hyde, D. M. and Hollinger, M. A. (1993) Effect of antibody to transforming growth factor β on bleomycin induced accumulation of lung collagen in mice. Thorax 48:959-966.

Goraca, A. and Skibska, B. (2008) Beneficial effect of alpha-lipoic acid on lipopolysaccharide-induced oxidative stress in bronchoalveolar lavage fluid. J. Physiol. Pharmacol. 59(2):379-86.

Griffith, O. W. (1980) Determination of glutathione and glutathione disulfide using glutathione reductase and 2-vinylpyridine. Anal. Biochem. 106(1):207-212.

Hackett, T. L., Holloway, R., Holgate, S. T. and Warner, J. A. (2008) Dynamics of pro-inflammatory and anti-inflammatory cytokine release during acute inflammation in chronic obstructive pulmonary disease: an ex vivo study. Respir. Res. 9(1):doi: 10.1186/1465-9921-9-47.

Han, S. G., Castranova, V. and Vallyathan, V. (2007) Comparative cytotoxicity of cadmium and mercury in a human bronchial epithelial cell line (BEAS-2B) and its role in oxidative stress and induction of heat shock protein. J. Toxicol. Environ. Health A. 70(10):852-860.

Helander, I., Saxen, H., Salinoja-Salonen, M. and Rylander, R. (1982) Pulmonary toxicity of endotoxins: comparison of lipopolysaccharides from various bacterial species. Infect. Immun. 25:528-532.

Hermanns, M. I., Unger, R. E., Kehe, K., Peters, K. and Kirkpatrick, C. J. (2004) Lung epithelial cell lines in coculture with human pulmonary microvascular endothelial cells: development of an alveolo-capillary barrier in vitro. Laboratory Investigation) 84:736-752.

Herseth, J. I., Volden, V., Schwarze, P. E., Låg, M. and Refsnes, M. (2008) IL-1beta differently involved in IL-8 and FGF-2 release in crystalline silica-treated lung cell co-cultures. Part. Fibre Toxicol. 2008 5:16 doi:10.1186/1743-8977-5-16.

Hoogerwerf, J. J., de Vos, A. F., Bresser, P., van der Zee, J. S., Pater, J. M., de Boer, A., Tanck, M., Lundell, D. L., Her-Jenh, C., Draing, C, von Aulock, S. and van der Poll, T. (2008) Lung inflammation induced by lipoteichoic acid or lipopolysaccharide in humans. Am. J. Respir. Crit. Care Med. 178(1):34-41.

Hoshino, T., Okamoto, M., Sakazaki, Y., Kato, S., Young, H. A. and Aizawa, H. (2009) Role of proinflammatory cytokine IL-18 and IL-1 {beta} in bleomycin-induced lung injury in humans and mice. Am. J. Respir. Cell. Mol. Biol. doi:10.1165/rcmb.2008-0182OC.

Imler, J. L. and Zheng, L. (2004) Biology of Toll receptors: Lessons from insects and mammals. J. Leukoc. Biol. 75:18-26.

Jarup, L., Berglund, M., Elinder, C. G., Nordberg, G. and Vahter, M. (1998) Health effects ofcadmium exposure: a review of the literature and a risk estimate. Scand. J. Work. Environ. Health. 24:1-51.

Jin, T., Lu, J. and Nordberg, M. (1998) Toxicokinetics and biochemistry of cadmium with special emphasis on the role of metallothionein. Neurotoxicology. 19:529-535.

Johnston, C. J., Finkelstein, J. N., Gelein, R. and Oberdörster, G. (1998) Pulmonary cytokine and chemokine mRNA levels after inhalation of lipopolysaccharide in C57BL/6 mice. Tox. Sci. 46:300-307.

Joseph, P. (2009) Mechanisms of cadmium carcinogenesis. Toxicol. Appl. Pharmacol. 238(3):272-279.

Karmiol, S., Remick, D. G., Kunkel, S. L., Phan, S. L. (1993) Regulation of rat pulmonary endothelial cell interleukin-6 production by bleomycin: effects of cellular fatty acid composition. Am. J. Respir. Cell Mol. Biol. 9:628-636.

Kasper, M., Seidel, D., Knels, L., Morishima, N., Neisser, A., Bramke, S. and Koslowski, R. (2004) Early signs of lung fibrosis after in vitro treatment of rat lung slices with CdCl2 and TGF-beta1. Histochem. Cell Biol. 121(2):131-140.

Kataranovski, M., Kataranovski, D., Savic, D., Jovcic, G., Bogdanovic, Z., and Jovanovic, T. (1998) Granulocyte and plasma cytokine activity in acute cadmium intoxication in rats. Physiol. Res. 47(6):453-461.

Ke, S., Rabson, A. B., Germino, J. F., Gallo, M. A. and Tian, Y. (2001) Mechanism of suppression of cytochrome P-450 1A1 expression by tumor necrosis factor-alpha and lipopolysaccharide. J. Biol. Chem. 276(43):39638-39644.

Keizer, H. G., Pinedo, H. M., Schuurhui, G. J. and Joenje, H. (1990) Doxorubicin (adriamycin): a critical review of free radical-dependent mechanisms of cytotoxicity. Pharmacol. Ther. 47:219-231.

Kollef, M. H. and Schuster, D. P. (1995) The acute respiratory distress syndrome. N. Engl. J. Med. 332:27-37.

Kundu, S., Sengupta, S., Chatterjee, S., Mitra, S. and Bhattacharyya, A. (2009) Cadmium induces lung inflammation independent of lung cell proliferation: a molecular approach. J. Inflamm. (Lond). 6(19):doi: 10.1186/1476-9255-6-19.

Lehnert, N. M., Gary, R. K., Marrone, B. L. and Lehnert, B. E. (2001) Inhibition of normal human lung fibroblast growth by beryllium. Toxicology. 160(1-3):119-27.

Lin, C. J., Yang, P. C., Hsu, M. T., Yew, F. H., Liu, T. Y., Shun, C. T., Tyan, S. W. and Lee, T. C. (1998)_Induction of pulmonary fibrosis in organ-cultured rat lung by cadmium chloride and transforming growth factor-beta1._{—}Toxicology. 127(1-3):157-166.

Manoury, B., Nenan, S., Leclerc, O., Guenon, I., Boichot, E., Planquois, J. M., Bertrand, C. P. and Lagente, V. (2005) The absence of reactive oxygen species production protects mice against bleomycin-induced pulmonary fibrosis. Respir. Res. 6:11.

Martin, C. J., Antonini, J. and Doney, B. C. (2009) A case report of elevated blood cadmium. Occup Med (Lond). 59(2): 130-132.

McLemore, T. L., Adelberg, S., Liu, M. C., McMahon, N. A., Yu, S. J., Hubbard, W. C., Czerwinski, M., Wood, T. G., Storeng, R., Lubet, R. A., Eggleston, J. C., Boyd, M. R. and Hines, R. N. (1990) Expression of CYP1A1 gene in patients with lung cancer: evidence for cigarette smoke-induced gene expression in normal lung tissue and for altered gene regulation in primary pulmonary carcinomas. J. Natl. Cancer Inst. 82(16): 1333-1339.

Meng, Q., Walker, D. M., McDonald, J. D., Henderson, R. F., Carter, M. M., Cook Jr, D. L., McCash, C. L., Tones, S. M., Bauer, M. J., Seilkop, S. K., Upton, P. B., Georgieva, N. I., Boysen, G., Swenberg, J. A. and Walker, V. E. (2007) Age-, gender-, and species-dependent mutagenicity in T cells of mice and rats exposed by inhalation to 1,3-butadiene. Chem. Biol. Interact. 166(1-3):121-131.

Micallef, M., Hosokawa, M., Togashi, Y. and Kobayashi, H. (1992) Rat macrophage activation after treatment with the bleomycin group of antitumour antibiotics in vivo. Cancer Immunol. Immunother. 35:106-112.

Minchin, R. F., Johnston, M. R., Schuller, H. M., Aiken, M. A. and Boyd, M. R. (1988) Pulmonary toxicity of doxorubicin administered by in situ isolated lung perfusion in dogs. Cancer 61(7):1320-1325.

Minotti, G., Menna, P., Salvatorelli, E., Cairo, G. and Gianni, L. (2004) Anthracyclines: molecular advances and pharmacologic developments in antitumor activity and cardiotoxicity. Pharmacol. Rev. 56:185-229.

Monshouwer, M., Witkamp, R. F., Nijmeijer, S. M., Van Leengoed, L. A., Vernooy, H. C., Verheijden, J. H. and Van Miert, A. S. (1996) A lipopolysaccharide-induced acute phase response in the pig is associated with a decrease in hepatic cytochrome P450-mediated drug metabolism. J. Vet. Pharmacol. Ther. 19(5):382-388.

Moseley, P. L., Hemken, C. and Hunninghake, G. W. (1986) Augmentation of fibroblast proliferation by bleomycin. J. Clin. Invest. 78:1150-1154.

Nandi, M., Slone, D., Jick, H., Shapiro, S. and Lewis, G. P. (1969) Cadmium content of cigarettes. Lancet. 2:329-330.

Otterson, G. A., Villalona-Calero, M. A., Sharma, S., Kris, M. G, Imondi, A., Gerber, M., White, D. A., Ratain, M. J., Schiller, J. H., Sandler, A., Kraut, M., Mani, S. and Murren, J. R. (2007) Phase I study of inhaled Doxorubicin for patients with metastatic tumors to the lungs. Clin. Cancer Res. 13(4):1246-52.

Ovrevik, J., Lag, M., Holme, J. A., Schwarze, P. E. and Refsnes, M. (2009) Cytokine and chemokine expression patterns in lung epithelial cells exposed to components characteristic of particulate air pollution. Toxicology 259(1-2):46-53.

Patwardhan, J. R. and Finckh, E. S. (1975) Fatal cadmium-fume pneumonitis. Med. J. Aust. 1(25):962-966.

Peretz, A., Checkoway, H., Kaufman, J. D., Trajber, I. and Lerman, Y. (2006) Silica, silicosis, and lung cancer. Isr. Med. Assoc. J. 8:114-118.

Phan, S. H. and Kunkel, S. L. (1992) Lung cytokine production in bleomycin induced pulmonary fibrosis. Exp. Lung Res. 18:18-43.

Piguet, P. F., Collart, M. A., Grau, G. E., Kapanci, Y., Vassalli, P. (1989) Tumor necrosis factor/cachectin plays a key role in bleomycin-induced pneumopathy and fibrosis. J. Exp. Med. 170:655-663.

Pugin, J., Schürer-Maly, C. C., Leturcq, D., Moriarty, A., Ulevitch, R. J. and Tobias, P. S. (1993) Lipopolysaccharide activation of human endothelial and epithelial cells is mediated by lipopolysaccharide-binding protein and soluble CD14. Proc Natl Acad Sci U S A. 90(7):2744-2748.

Renton, K. W., Dibb, S. and Levatte, T. L. (1999) Lipopolysaccharide evokes the modulation of brain cytochrome P4501A in the rat. Brain Res. 842(1):139-147.

Rocksen, D., Ekstrand-Hammarström, B., Johansson, L. and Bucht, A. (2003) Vitamin E reduces transendothelial migration of neutrophils and prevents lung injury in endotoxin-induced airway inflammation. Am. J. Respir. Cell Mol. Biol. 28:199-207.

Rylander, R., Snella, M. C. and Garcia, I. (1975) Pulmonary cell response patterns after exposure to airborne bacteria. Scand. J. Respir. Dis. 56:195-200.

Saitoh, T., Kokue, E. and Shimoda, M. (1999) The suppressive effects of lipopolysaccharide-induced acute phase response on hepatic cytochrome P450-dependent drug metabolism in rabbits. J. Vet. Pharmacol. Ther. 22(2):87-95.

Sakaguchi, T., Sakaguchi, S., Nakamura, I. and Kagami, M. (1984) Effects of beryllium chloride on cultured cells. Sangyo Igaku. 26(3):224-229.

Sato, E., Koyama, S., Masubushi, T., Takamizawa, A., Kubo, K., Nagai, S. and Izumi, T. (1999) Bleomycin stimulates lung epithelial cells to release neutrophil and monocyte chemotactic activities. Am. J. Physiol. Lung Cell Mol. Physiol. 276: L941-L950.

Scheule, R. K., Perkins, R. C., Hamilton, R., Holian, A. (1992) Bleomycin stimulation of cytokine secretion by the human alveolar macrophage. Am. J. Physiol. 262(4 Pt 1):L386-391.

Schmidt, J. A., Mizel, S. B., Cohen, D. and Green, I. (1982) Interleukin-1: a potential regulator of fibroblast proliferation. J. Immunol. 128:2177-2182.

Sheehan, D. C. and Hrapchak, B. B. (1980) Theory and Practice of Histotechnology, 2^{nd} Edition, CV. Mosby Co., St. Louis Mo.

Shin, D. H. (1996) Expression of IL-6 mRNA by Human Lung Fibroblast Cell Line Treated with Cadmium. Kor. J. Occup. Med. 8(1):34-42.

Snell, J. D. (1966) Effects of inhaled endotoxin. J. Lab. Clin. Med. 67:624-632.

Snella, M. C. (1981) Effects of bacterial endotoxin inhalation. Rev. Epidemiol. Sante Pub. 29:209-216.

Sprince, N. L, Kazemi, H. and Hardy, H. L. Current (1976) Problems of differentiating between beryllium disease and sarcoidosis. Ann. N. Y. Acad. Sci. 278:654-665.

Sugerman, B. J., Aggarwal, B B, Figari, I. S. Palladino Jr., M. A. and Shepard, H. M. (1985) Recombinant human tumor necrosis factor α: effects on proliferation of normal and transformed cells in vitro. Science 230:943-945.

Takamizawa, A., Koyama, S., Sato, E., Masubushi, T., Kubo, K., Sekiguchi, M., K., Nagai, S. and Izumi, T. (1999) Bleomycin stimulates lung fibroblasts to release neutrophil and monocyte chemotactic Activity. J. Immunol. 162: 6200-6208.

Tan, C., Tasaka, H., Yu, K. P., Murphy, M. L. and Karnofsky, D. A. (1967) Daunomycin, An Antitumor Antibiotic, In the Treatment of Neoplastic Disease. Clinical Evaluation with Special Reference to Childhood Leukemia. Cancer 20:333-353.

Tinkle, S. S. and Newman, L. S. (1997) Beryllium-stimulated release of tumor necrosis factor-alpha, interleukin-6, and their soluble receptors in chronic beryllium disease. Am. J. Respir. Crit. Care Med. 156(6):1884-1891.

Tinkle, S. S., Schwitters, P. W. and Newman, L. S. (1996) Cytokine production by bronchoalveolar lavage cells in chronic beryllium disease. Environ. Health. Perspect. 104(Suppl 5):969-971.

Vandesompele, J., De Preter, K., Pattyn, F., Poppe, B., Van Roy, N., De Paepe, A. and Speleman, F. (2002) Accurate normalization of real-time quantitative RT-PCR data by geometric averaging of multiple internal control genes. Genome Biol. 3(7):research0034.

Vanhée, D., Gosset, P., Marquette, C. H., Wallaert, B., Lafitte, J.J., Gosselin, B., Voisin, C. and Tonnel, A. B. (1995) Secretion and mRNA expression of TNF alpha and IL-6 in the lungs of pneumoconiosis patients. Am. J. Respir. Crit. Care Med. 152(1):298-306.

Ware, L. B., and M. A. Matthay. (2000) The acute respiratory distress syndrome. N. Engl. J. Med. 342:1334-1349.

Yang, C. F., Shen, H. M., Shen, Y., Zhuang, Z. X. and Ong, C. N. (1997) Cadmium-induced oxidative cellular damage in human fetal lung fibroblasts (MRC-5 cells). Environ Health Perspect. 105(7): 712-716.

Zhang, K., Gharaee, M., McGarry, B., Remick, D. and Phan, S. H. (1997) TNF-α mediated lung cytokine networking and eosinophil recruitment in pulmonary fibrosis. J. Immunol. 158:954-959.

Zordoky, B. N. and El-Kadi, A. O. (2008) Induction of several cytochrome P450 genes by doxorubicin in H9c2 cells. Vascul. Pharmacol. 49(4-6):166-172.

What is claimed is:

1. A method for predicting the in vivo respiratory toxicity of a compound, comprising:
(a) culturing mammalian cells;
(b) contacting the mammalian cells with a concentration of the compound;
(c) measuring the expression level of one or more marker genes in the mammalian cells after contacting the cells with the compound;
(d) monitoring multiple endpoints of cell viability and general cell health;
(e) conducting a computational analysis of the concentration of the compound used to contact the cells, and the measured expression level(s) of the one or more marker genes; and
(f) determining a predicted in vivo respiratory toxicity value based on the computational analysis.

2. The method of claim 1, wherein the mammalian cells are human cells selected from the group: three dimensional synthetic airway models derived from epithelial cells from tracheal or bronchial tissue, or both; lung cells, NCI-H460, NCI-H549, NCI-H661, NCI-H292, BEAS-2B; Clara cell lines; Clara cells in culture; precision cut tissue slices of lung;
and combination cultures thereof

3. The method of claim 1, wherein the cells are contacted with varying dosages of compound.

4. The method of claim 1, wherein the marker gene(s) are selected from the group consisting of: CYP1A1; Bax; Bcl2; TNFα; TGFβ; IL-1a; IL-6; and IL-8; quinone reductase; CD-86; aldo-keto reductase; thioredoxin; and thioredoxin reductase.

5. The method of claim 1, wherein the multiple endpoints comprise at least one of cellular structure, cell viability, and oxidative stress.

6. The method of claim 1, wherein the computational analysis comprises data from a set of known lung or respiratory toxicants and known exposure levels and toxicity categories.

7. A method for screening a compound for in vivo respiratory toxicity comprising:
(a) culturing mammalian cells;
(b) contacting the mammalian cells with a concentration of the compound;
(c) measuring the expression level of one or more marker genes in the mammalian cells after contacting the cells with the compound;
(d) monitoring multiple endpoints of cell viability and general cell health;
(e) conducting a computational analysis of the concentration of the compound used to contact the cells, and the measured expression level(s) of the one or more marker genes;
(f) determining a predicted in vivo respiratory toxicity value based on the computational analysis; and
(g) determining whether the toxicity value of the compound falls within acceptable limits for the particular in vivo use.

8. The method of claim 7, wherein the mammalian cells are human cells selected from the group: three dimensional synthetic airway models derived from epithelial cells from tracheal or bronchial tissue, or both; lung cells, NCI-H460, NCI-H549, NCI-H661, NCI-H292, BEAS-2B; Clara cell lines; Clara cells in culture; precision cut tissue slices of lung; and combination cultures thereof

9. The method of claim 7 wherein the cells are contacted with varying dosages of compound.

10. The method of claim 7, wherein the marker gene(s) are selected from the group consisting of: quinone reductase; CYP1A1; Bax; Bcl2; TNFα; TGFβ; IL-1a; IL-6; and IL-8; CD-86; aldo-keto reductase; thioredoxin; and thioredoxin reductase.

11. The method of claim 7, wherein the multiple endpoints comprise at least one of cellular structure, cell viability, and oxidative stress.

12. The method of claim 7, wherein the computational analysis comprises data from a set of known lung or respiratory toxicants and known exposure levels and toxicity categories.

13. A method for categorizing the in vivo respiratory toxicity of a compound, comprising:
(a) culturing mammalian cells;
(b) contacting the mammalian cells with a concentration of the compound;
(c) measuring the expression level of one or more marker genes in the mammalian cells after contacting the cells with the compound;
(d) monitoring multiple endpoints of cell viability and general cell health;
(e) conducting a computational analysis of the concentration of the compound used to contact the cells, and the measured expression level(s) of the one or more marker genes; and
(f) determining a predicted in vivo respiratory toxicity value based on the computational analysis;
wherein the computational analysis (e) comprises a comparison of the data from the compound with data gathered from at least two compounds with known respiratory toxicity profiles,
wherein the two compounds with known respiratory toxicity profiles are classified as a respiratory sensitizer, a respiratory irritant, or a respiratory corrosive, wherein the at least two compounds are not members of the same toxicity profile class.

14. The method of claim 13, wherein the mammalian cells are human cells selected from the group: three dimensional synthetic airway models derived from epithelial cells from tracheal or bronchial tissue, or both; lung cells, NCI-H460, NCI-H549, NCI-H661, NCI-H292, BEAS-2B; Clara cell lines; Clara cells in culture; precision cut tissue slices of lung; and combination cultures thereof

15. The method of claim 13, wherein the cells are contacted with varying dosages of compound.

16. The method of claim 13, wherein the marker gene(s) are selected from the group consisting of: quinone reductase; CYP1A1; Bax; Bcl2; TNFα; TGFβ; IL-1a; IL-6; and IL-8; CD-86; aldo-keto reductase; thioredoxin; and thioredoxin reductase.

17. The method of claim 13, wherein the multiple endpoints comprise at least one of cellular structure, cell viability, and oxidative stress.

18. The method of claim 13, wherein the computational analysis comprises data from a set of known lung or respiratory toxicants and known exposure levels and toxicity categories.

19. A kit comprising reagents for
(a) reagents for measuring the expression level of one or more marker genes in mammalian cells in culture;
(b) reagents for monitoring multiple endpoints of cell viability and general cell health;
(c) optional software for executing instructions on a CPU that performs a computational analysis of the measured expression level(s) of the one or more marker genes, and the cell viability and general cell health data; and
(d) instructions for use of the kit.
